(19) United States
(12) Patent Application Publication
(10) Pub. No.: US 2007/0299067 A1
(43) Pub. Date: Dec. 27, 2007

(54) Quinoline and isoquinoline derivatives as phosphodiesterase 10 inhibitors

(76) Inventors: Ruiping Liu, Huntington, NY (US); Mark P. Arrington, Westwood, NJ (US); Allen T. Hopper, Mahwa, NJ (US); Richard D. Conticello, Cortlandt Manor, NY (US); Stephen A. Hitchcock, Westlake Village, CA (US)

Correspondence Address: JONES DAY, 222 EAST 41ST ST, NEW YORK, NY, 10017, US

(21) Appl. No.: 11/716,307

(22) Filed: Mar. 8, 2007

Related U.S. Application Data
(60) Provisional application No. 60/780,611, filed on Mar. 8, 2006.

(51) Int. Cl. A61K 31/535; A61K 31/47; A61P 25/00; C07D 217/00; C07D 217/22; C07D 413/00

(52) U.S. Cl. 514/233.5; 514/307; 514/310; 514/313; 544/128; 546/139; 546/141; 546/159

(57) ABSTRACT

The present invention is directed to certain quinoline and isoquinoline compounds that are PDE10 inhibitors, pharmaceutical compositions containing such compounds and processes for preparing such compounds. The invention is also directed to methods of treating diseases mediated by PDE10 enzyme, such as obesity, non-insulin dependent diabetes, schizophrenia, bipolar disorder, obsessive-compulsive disorder, and the like.

### CROSS-REFERENCE

This application claims the benefit of U.S. Provisional Application No. 60/780,611, filed Mar. 8, 2006, the disclosure of which is incorporated herein by reference in its entirety.

### FIELD OF THE INVENTION

Provided herein are certain quinoline and isoquinoline compounds that are PDE10 inhibitors, pharmaceutical compositions containing such compounds, and processes for preparing such compounds. Provided herein also are methods of treating disorders or diseases treatable by inhibition of PDE 10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activities.

Cyclic nucleotides are produced from the actions of adenylyl cyclase and guanylyl cyclase, which convert ATP to cAMP and GTP to cGMP. Extracellular signals, often through the actions of G protein-coupled receptors, regulate the activities of the cyclases. Alternatively, the amount of cAMP and cGMP may be altered by regulating the activities of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE1 is stimulated by Ca²⁺/calmodulin. PDE2 activity is stimulated by cGMP. PDE3 is inhibited by cGMP. PDE4 is cAMP specific and is specifically inhibited by rolipram. PDE5 is cGMP-specific. PDE6 is expressed in retina.

PDE10 sequences were identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because at least three alternative exons encode N termini and two exons encode C-termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE 10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunoprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, for example, in neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein is a compound of Formula (I): 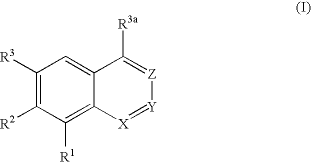
or an individual stereoisomer, a mixture of stereoisomers, or a pharmaceutically acceptable salt or solvate thereof, wherein:
X is nitrogen and Y and Z are each —CH═ or one of Y and Z is nitrogen and the other is —CH═ and X is —CR═ (where R is hydrogen, alkyl, halo, or cyano);
R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, cycloalkyl, cycloalkyloxy, cycloalkylalkyloxy, hydroxyalkyl, hydroxyalkyloxy, alkoxyalkyl, alkoxyalkyloxy, -(alkylene)-NR¹³R¹⁴ and —O-alkylene)-NR¹⁵R¹⁶ (where R¹³, R¹⁴, R¹⁵, and R¹⁶ are independently hydrogen or alkyl), wherein one or two carbon atoms in the alkyl chain in hydroxyalkyl, hydroxyalkyloxy, alkoxyalkyl, alkoxyalkyloxy, -(alkylene)-NR¹³R¹⁴ or —O-(alkylene)-NR¹⁵R¹⁶ are optionally replaced by one to two oxygen or nitrogen atom(s), and provided that at least one of R¹, R², and R³ is not hydrogen; and
R^{3a} is aryl, heteroaryl, or heterocyclyl ring substituted with:
   R⁴, where R⁴ is hydrogen, alkyl, halo, haloalkyl, haloalkoxy, cycloalkyl, cycloalkenyl, cycloalkylalkyl, aryl, aralkyl, heteroaryl, heteroaralkyl, heterocyclyl, heterocyclylalkyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹, and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶, where R⁵ and R⁶ are each independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl, and provided that at least one of R⁴, R⁵, and R⁶ is not hydrogen;
   wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl or optionally substituted heterocyclyl; and additionally substituted with one or two substitutents independently selected from R^{d} and R^{e} where R^{d} and R^{e} are hydrogen or fluoro;
provided that:

(a) when R is hydrogen, R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, and cycloalkyl, and:
(i) R^{3a} is pyrrolidin-1-yl, then R⁴ is not —X¹R⁷, where X¹ is —O—, and R⁷ is substituted or unsubstituted, aryl or heteroaryl;
(ii) R^{3a} is piperidin-1-yl, where one of R⁴, R⁵ and R⁶ is hydrogen and another of R⁴, R⁵ and R⁶ is substituted or unsubstituted aryl or heteroaryl, then the remaining member of R⁴, R⁵ and R⁶ is not hydrogen; alkyl; carboxy; cyano; hydroxyl; alkoxy; —COR′, —CONR′R″ or —NR′R″ (where R′ and R″ are independently hydrogen, alkyl, or unsubstituted aryl); or —NHCOR′ (where R′ is alkyl or unsubstituted aryl); or
(iii) R^{3a} is piperidin-1-yl, where two of R⁴, R⁵ and R⁶ are hydrogen, then remaining of R⁴, R⁵ and R⁶ is not —COR′ (where R¹ is alkyl or unsubstituted aryl), —COOR¹ (where R¹ is alkyl or unsubstituted aryl), —CONR′R″, —NR′R″ or —NHCOR′ (where each R″ is hydrogen, alkyl, or unsubstituted aryl, and each R′ is unsubstituted aryl);

(b) when R is hydrogen, R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, and cycloalkyl, then:
(i) R^{3a} is not substituted or unsubstituted 1,2,3,4-tetrahydroisoquinolin-3-yl or 1,2,3,4-tetrahydroisoquinolin-2-yl; and
(ii) R^{3a} is not monosubstituted or disubstituted pyrrolidinyl where the one or two substituents are alkyl;

(c) When R is hydrogen, alkyl, or alkoxy, R¹, R², and R³ are independently hydrogen, halo, haloalkyl, alkyl, alkoxy, carboxy, hydroxymethyl or hydroxy, and R³a is aryl, then one of R⁴, R⁵, and R⁶ is an aromatic or alicyclic ring or a group that contains an aromatic or alicyclic ring provided that the aromatic or alicyclic ring is not phenyl (optionally substituted with one, two, or three substitutents, independently selected from cyano, halo, —CONH₂ and haloalkyl), benzyl, benzyloxy, 1H-benzimidazol-2-ylthio, 1H-benzimidazol-2-ylsulfinyl, pyridinyl (optionally substituted with halo or —CONH₂), pyrimidinyl, or morpholin-4-yl-carbonyl;

(d) when R is hydrogen, R¹, R², and R³ are independently hydrogen, halo, alkoxy, or hydroxy, and R^{3a} is heteroaryl, then the heteroaryl ring is not phthalazin-1-yl optionally substituted with R⁴, R⁵ and R⁶, where R⁴ is alkyl, and R⁵ and R⁶ are alkoxy;

isoquinolinyl optionally substituted with one or two substituents selected from alkoxy and hydroxy;

1H-indolyl optionally substituted with R⁴, R⁵, and R⁶ where R⁴ is hydrogen, one of R⁵ and R⁶ is hydrogen, alkyl, or alkoxy, and other of R⁵ and R⁶ is alkyl, alkoxy, haloalkyl, dialkylaminoalkyl, or hydroxyalkyl;

benzo[c]isoxazolyl optionally substituted with R⁴, R⁵, and R⁶ where one of R⁴, R⁵, and R⁶ is hydrogen and the other two of R⁴, R⁵, and R⁶ are independently selected from alkoxy, aryl, and benzyloxy;

1H-indazolyl optionally substituted with one or two of alkoxy or hydroxy;

pyrrolyl substituted with R⁴, R⁵, and R⁶ where one of R⁴, R⁵, and R⁶ is hydrogen or alkyl and the other two of R⁴, R⁵, and R⁶ are phenyl optionally substituted with one or two alkoxy;

thienyl optionally substituted with halo; or

pyrazolyl optionally substituted with R⁴, R⁵, and R⁶ where R⁴ is hydrogen, one of R⁵ and R⁶ is alkoxycarbonyl and the other of R⁵ and R⁶ is alkoxyalkyl;

(e) when R is hydrogen or alkoxy, R¹, R², and R³ are independently hydrogen, halo, alkyl, haloalkyl, haloalkoxy, alkoxy, carboxy, hydroxymethyl or hydroxy, then R^{3a} is not:
monosubstituted piperazinyl [wherein the substitutent on the piperazinyl ring is alkyl, alkoxycarbonyl, phenyl, —COR′ (where R′ is alkyl; or piperidinyl or pyrrolidinyl each optionally substituted with one or two substituents each independently selected from alkyl or hydroxyl), hydroxyalkyl, —CONHR′ (where R′ is phenyl substituted with fluoro or phenoxy), 1H-benzo[d]imidazol-2(3H)-one optionally substituted with alkyl, or 3,4-dihydroquinolinyl-2(1H)-one];

substituted or unsubstituted benzimidazolyl, 1,2,3,4-tetrahydroisoquinolinyl, isoquinolinyl, isobenzofuranyl-1(3H)-one, 1,2,3-oxadiazolyl-5(2H)-one, 1,3,4-oxadiazolyl-2(3H)-one, 2,3-dihydrobenzo[b][1,4]dioxinyl, benzo[d][1,3]dioxolyl, 1,2,4,5,6,7-hexahydropyrazolo[1,5-a]pyridinyl, 1,2-dihydropyrazolo[1,5-a]pyridinyl, H-pyrazolo[1,5-a]pyridinyl, 5,6-dihydro-4H-pyrrolo[1,2-b]pyrazolyl, benzisoxazolyl, 1,1-dioxo-3H-benzo[c][1,2]oxathiolyl, benzofuranyl-2(3H)-one, (Z)-1H-benzo[e][1,4]diazepinyl-2(3H)-one, 1,3a-dihydropyrazolo[1,5-a]pyridinyl, oxazolyl-2(3H)-one, naphthyl, or imidazo[5,1-a]isoquinolinyl;

mono or disubstituted piperidinyl (where one substituent is hydrogen or hydroxy, and the other substitutent is alkoxy, hydroxyl, carboxy, or 1H-benzo[d]imidazol-2(3H)-one optionally substituted with alkyl); or pyrrolidinyl optionally substituted with alkyl or alkoxy; and

(f) when X is N, then at least two of R′, R² and R³ are not simultaneously hydrogen; and

(g) the compound is not a salt of (a)-(f).

In a second aspect, provided herein is a pharmaceutical composition comprising a compound of Formula (I) or a pharmaceutically acceptable salt thereof and a pharmaceutically acceptable expicient.

In a third aspect, this invention is directed to a method of treating a disorder treatable by inhibition of PDE10 in a patient which method comprises administering to the patient a pharmaceutical composition comprising a compound of Formula (I) or a pharmaceutically acceptable salt thereof and a pharmaceutically acceptable excipient. Within this aspect, the disease is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomer, mixtures of stereoisomers where the compound of Formula (I) has at least a stereochemical centre), a pharmaceutically acceptable salt thereof, or mixtures thereof.

### DETAILED DESCRIPTION

### Definitions

Unless otherwise stated, the following terms used in the specification and claims are defined for the purposes of this Application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated, e.g., methylene, ethylene, propylene, 1-methylpropylene, 2-methylpropylene, butylene, pentylene, and the like.

“Alkylthio” means a —SR radical, where R is alkyl as defined above, e.g., methylthio, ethylthio, and the like.

“Alkylsulfinyl” means a —SOR radical where R is alkyl as defined above, e.g., methylsulfinyl, ethylsulfinyl, and the like.

“Alkylsulfonyl” means a —SO₂R radical, where R is alkyl as defined above, e.g., methylsulfonyl, ethylsulfonyl, and the like.

“Amino” means an —NH₂.

“Alkylamino” means an —NHR radical, where R is alkyl as defined above, e.g., methylamino, ethylamino, propylamino, or 2-propylamino, and the like.

“Alkoxy” means an —OR radical, where R is alkyl as defined above, e.g., methoxy, ethoxy, propoxy, or 2-propoxy, n-, iso-, or tert-butoxy, and the like.

“Alkoxycarbonyl” means a —C(O)OR radical, where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means an —OR radical, where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two —NRR′, where R is hydrogen, alkyl, or —COR^{a}, where R^{a} is alkyl, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical, where R is aminoalkyl as defined above, e.g., 2-aminoethoxy, 2-dimethylaminopropoxy, and the like.

“Aminocarbonyl” means a —CONRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminocarbonyl, 2-dimethylaminocarbonyl, and the like.

“Aminosulfinyl” means a —SONRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminosulfinyl, 2-dimethylaminosulfinyl, and the like.

“Aminosulfonyl” means a —SO₂NRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetyl, propionyl, benzoyl, pyridinylcarbonyl, and the like. When R in a —COR radical is alkyl, the radical is also referred to herein as “alkylcarbonyl.”

“Acylamino” means an —NHCOR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetylamino, propionylamino, and the like.

“Aryl” means a monovalent monocyclic or bicyclic aromatic hydrocarbon radical of 6 to 12 ring atoms, e.g., phenyl or naphthyl.

“Aralkyl” means an -(alkylene)-R radical, where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl. Additionally, one or two ring carbon atoms may optionally be replaced with a —CO— group.

“Cycloalkenyl” means a cyclic nonaromatic monovalent bridged or non-bridged hydrocarbon radical of five to ten carbon atoms, which contains at least one carbon-carbon double bond, e.g., cyclopentenyl or cyclohexenyl. Additionally, one or two ring carbon atoms may optionally be replaced by a —CO— group.

“Cycloalkylalkyl” means an -(alkylene)-R radical, where R is cycloalkyl as defined above; e.g., cyclopropylmethyl, cyclobutylmethyl, cyclopentylethyl, or cyclohexylmethyl, and the like.

“Cycloalkyloxy” means an —OR radical, where R is cycloalkyl as defined, e.g., cyclopropyloxy, cyclobutyloxy, cyclopentyloxy, cyclohexyloxy, and the like.

“Cycloalkylalkyloxy” means an —OR radical, where R is cycloalkylalkyl as defined, e.g., cyclopropylmethyloxy, cyclobutylmethyloxy, cyclopentylethyloxy, cyclohexylmethyloxy, and the like.

“Carboxy” means —COOH.

“Disubstituted amino” means an —NRR′ radical, where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., dimethylamino, phenylmethylamino, and the like.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means an —OR radical, where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that, if two hydroxy groups are present, they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical, where R is hydroxyalkyl as defined above.

“Heterocyclyl” means a saturated or unsaturated monovalent monocyclic group of 4 to 8 ring atoms, in which one or two ring atoms are heteroatom(s), independently selected from N, O, and S(O)ₙ, where n is an integer from 0 to 2, the remaining ring atoms are C. Additionally, one or two ring carbon atoms can optionally be replaced by a —CO— group, and the heterocyclic ring may be fused to phenyl or heteroaryl ring, provided that the entire heterocyclyl ring is not completely aromatic. Unless stated otherwise, the fused heterocyclyl ring can be attached at any ring atom. More specifically, the term “heterocyclyl” includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring has five, six or seven ring atoms, and is not fused to phenyl or heteroaryl ring, it is referred to herein as “monocyclic five-, six-, or seven-membered heterocyclyl ring, or five-, six-, or seven-membered heterocyclyl ring.” When the heterocyclyl ring is unsaturated, it can contain one or two ring double bonds, provided that the ring is not aromatic.

“Heterocyclylalkyl” means an -(alkylene)-R radical, where R is heterocyclyl ring as defined above, e.g., tetrahydrofuranylmethyl, piperazinylmethyl, morpholinylethyl, and the like.

“Heteroaryl” means a monovalent monocyclic or bicyclic aromatic radical of 5 to 10 ring atoms, where one or more, preferably one, two, or three, ring atoms are heteroatoms independently selected from N, O, and S, and the remaining ring atoms are carbon, e.g., benzofuranyl, benzo[d]thiazolyl, isoquinolinyl, quinolinyl, thiophenyl, imidazolyl, oxazolyl, quinolinyl, furanyl, thazolyl, pyridinyl, and the like.

“Heteroaralkyl” means an -(alkylene)-R radical, where R is heteroaryl as defined above.

“Monosubstituted amino” means an —NHR radical, where R is alkyl, acyl, sulfonyl, aryl, aralkyl, heteroaryl, heteroaralkyl, heterocyclyl, heterocyclylalkyl, cycloalkyl, cycloalkylalkyl, hydroxyalkyl, alkoxyalkyl, or aminoalkyl, each as defined above, e.g., methylamino, 2-phenylamino, hydroxyethylamino, and the like.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups by routine manipulation or in vivo. Prodrugs of compounds of Formula (I) include compounds wherein a hydroxy, amino, carboxylic, or a similar group is modified. Examples of prodrugs include, but are not limited to, esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol, or any group containing a nitrogen atom, these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for instance, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “pharmaceutically acceptable salt” also refers to salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are non-toxic. Additional information on suitable pharmaceutically acceptable salts can be found in Remington's Pharmaceutical Sciences, Gennaro, A. R. (Mack Publishing Company, 18th ed., 1995), which is incorporated herein by reference.

The compounds of the present invention may have one or more asymmetric centers. Compounds of the present invention containing an asymmetrically substituted atom may be isolated in an optically active, racemic, or diastereomeric form. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof, are within the scope of this invention.

Additionally, as used herein, the term “alkyl” includes all the possible isomeric forms of said alkyl group albeit only a few examples are set forth. Furthermore, when a cyclic group, such as aryl, heteroaryl, and heterocyclyl, is substituted, it includes all the positional isomers albeit only a few examples are set forth.

All polymorphic forms and solvates, including hydrates, of a compound of Formula (I) are also within the scope of this invention.

“Oxo” means the ═(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents, each independently selected from alkyl, halo, alkoxy, alkylthio, haloalkyl, haloalkoxy, amino, alkylamino, dialkylamino, hydroxy, cyano, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, and sulfonyl, each as defined herein.

“Optionally substituted heteroaryl” means a monovalent monocyclic or bicyclic aromatic radical of 5 to 10 ring atoms, where one or more, preferably one, two, or three ring atoms are heteroatoms, each independently selected from N, O and S, and the remaining ring atoms are carbon that is optionally substituted with one, two, or three substituents, each independently selected from alkyl, halo, alkoxy, alkylthio, haloalkyl, haloalkoxy, amino, alkylamino, dialkylamino, hydroxy, cyano, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, and sulfonyl, each as defined herein. More specifically, the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl, each optionally substituted as indicated above.

“Optionally substituted heterocyclyl” means a saturated or unsaturated monovalent cyclic group of 3 to 8 ring atoms in which one or two ring atoms are heteroatoms, each independently selected from N, O, and S(O)ₙ, where n is an integer from 0 to 2, and the remaining ring atoms are carbon. One or two ring carbon atoms can optionally be replaced by a —CO— group and is optionally substituted with one, two, or three substituents, each independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, and sulfonyl, each as defined herein.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical, where R is alkyl, haloalkyl, aryl, aralkyl, heteroaryl, heteroaralkyl, heterocyclyl, or heterocyclylalkyl, each as defined above, e.g., methylsulfinyl, phenylsulfinyl, benzylsulfinyl, and the like.

“Sulfonyl” means a —SO₂R radical, where R is alkyl, haloalkyl, aryl, aralkyl, heteroaryl, heteroaralkyl, heterocyclyl, or heterocyclylalkyl, each as defined above, e.g., methylsulfonyl, phenylsulfonyl, benzylsulfonyl, pyridinylsulfonyl, and the like.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity, and the age, weight, etc., of the mammal to be treated.

### Embodiments

In one aspect, provided herein is a compound of Formula (I), an individual stereoisomer, a mixture of stereoisomers, or pharmaceutically acceptable solvate or salt thereof, as defined in the Summary of the Invention.

(1) In one embodiment, X is nitrogen, and Y and Z are ═CH—.

(2) In another embodiment, Y is nitrogen, and X and Z are ═CH—.

(3) In yet another embodiment, Z is nitrogen, and X and Y are ═CH—.

(4) In yet another embodiment, Y is nitrogen, Z is —CH═, and X is —CR═, where R is alkyl.

(5) In another embodiment, Y is nitrogen, Z is —CH═, and X is —CR═, where R is methyl, ethyl, n-propyl, or isopropyl.

(6) In another embodiment, provided herein are compounds of Formula (I), wherein Y is nitrogen, Z is —CH═, and X is —CR═, where R is halo. Within this embodiment, one group of compounds of Formula (I) is that wherein R is fluoro or chloro.

(7) In yet another embodiment, Z is nitrogen, Y is —CH═, and X is —CR═ where R is alkyl.

(8) In another embodiment, Z is nitrogen, Y is —CH═, and X is —CR═ where R is methyl, ethyl, n- or iso-propyl.

(9) In yet another embodiment, Z is nitrogen, Y is —CH═, and X is —CR═ where R is halo. Within this embodiment, one group of compounds of Formula (I) is that wherein R is fluoro or chloro.

(A) Within the above embodiments 1-9, and subgroups contained therein, one group of compounds of Formula (I) is that wherein R¹ is hydrogen.

(B) Within the above embodiment 1-9, and subgroups contained therein, another group of compounds of Formula (I) is that wherein R¹ is hydrogen, R² is alkoxy, and R³ is cycloalkoxy or cycloalkylalkyloxy. Within this embodiment, one group of compounds is that wherein R² is methoxy, and R³ is cyclopropoxy, cyclobutyoxy, cyclopentoxy, or cyclohexyloxy. Within this embodiment, another group of compounds is that wherein R² is methoxy, and R³ is cycloproplmethyloxy, cyclopropylethoxy, cyclobutylmethyloxy, cyclobutylethyloxy, cyclopentylmethyloxy, cyclohexylmethyl, or cyclohexylethyloxy.

(C) Within the above embodiments 1-9, and subgroups contained therein, yet another group of compounds of Formula (I) is that wherein R¹ is hydrogen, R² is alkoxy, preferably methoxy or ethoxy, and R³ is hydroxyalkyl.

(D) Within the above embodiments 1-9, and subgroups contained therein, yet another group of compounds of Formula (I) is that wherein R¹ is hydrogen, R² is alkoxy, preferably methoxy or ethoxy, and R³ is hydroxyalkyloxy.

(E) Within the above embodiments 1-9, and subgroups contained therein, yet another group of compounds of Formula (I) is that wherein R¹ is hydrogen, R² is alkoxy, preferably methoxy or ethoxy, and R³ is alkoxyalkyl.

(F) Within the above embodiments 1-9, and subgroups contained therein, yet another group of compounds of Formula (I) is that wherein R¹ is hydrogen, R² is alkoxy, preferably methoxy or ethoxy, and R³ is alkoxyalkyloxy.

(G) Within the above embodiments 1-9, and subgroups contained therein, yet another group of compounds of Formula (I) is that wherein R¹ is hydrogen, R is alkoxy, preferably methoxy or ethoxy, and R³ is -(alkylene)-NR¹³R¹⁴, where R¹³ and R¹⁴ are as defined in the Summary of the Invention.

(H) Within the above embodiments 1-9, and subgroups contained therein, yet another group of compounds of Formula (I) is that wherein R¹ is hydrogen, R² is alkoxy, preferably methoxy or ethoxy, and R³ is —O-(alkylene)-NR¹⁵R¹⁶, where R¹⁵ and R¹⁶ are as defined in the Summary of the Invention

(I) Within the above embodiments 1-9, and subgroups contained therein, yet another group of compounds of Formula (I) is that wherein R¹ is hydrogen, R² is alkylamino, dialkylamino, fluoro, or trifluoromethoxy, and R³ is cycloalkoxy, cycloalkylalkyloxy, hydroxyalkoxy, alkoxylalkyloxy, or —O(alkylene)-NR¹⁵R¹⁶, where R¹⁵ and R¹⁶ are as defined in the Summary of the Invention

(J) Within the above embodiment 1-9, another group of compounds of Formula (I) is that wherein R¹ is hydrogen, and R² and R³ are alkoxy, preferably, methoxy or ethoxy.

(K) Within the above embodiments 1-9, another group of compounds of Formula (I) is that wherein R¹ is hydrogen, R² is alkoxy, and R³ is alkyl. Within this embodiment, one group of compounds of Formula (I) is that wherein R¹ is hydrogen, R² is methoxy or ethoxy, and R³ is methyl, ethyl, or propyl.

(L) Within the above embodiments 1-9, one group of compounds of Formula (I) is that wherein R¹ is hydrogen, R² is alkoxy, and R³ is cycloalkyl, preferably cyclopropyl. Within this embodiment, one group of compounds of Formula (I) is that wherein R is hydrogen, R¹ is methoxy or ethoxy, and R³ is cyclopropyl.

(M) Within the above embodiments 1-9, one group of compounds of Formula (I) is that wherein R¹ is hydrogen, R² is fluoro, trifluoromethoxy, methylamino, or dimethylamino, and R³ is alkyl, alkoxy, haloalkyl, halo, alkoxycarbonyl or cycloalkyl.

(N) Within the above embodiments 1-9, another group of compounds of Formula (I) is that wherein R¹ is hydrogen, R³is alkoxy, and R² is alkyl. Within this embodiment, one group of compounds of Formula (I) is that wherein R¹ is hydrogen, R³ is methoxy or ethoxy, and R² is methyl, ethyl, or propyl.

(O) Within the above embodiments 1-9, one group of compounds of Formula (I) is that wherein R¹ is hydrogen, R³ is alkoxy, and R² is cycloalkyl, preferably cyclopropyl. Within this embodiment, one group of compounds of Formula (I) is that wherein R¹ is hydrogen, R³ is methoxy or ethoxy and R² is cyclopropyl.

(P) Within the above embodiments 1-9, one group of compounds of Formula (I) is that wherein R¹ is hydrogen, one of R² and R³ is alkoxy and the other of R² and R³ is halo or haloalkoxy.

(i) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, one group of compounds of Formula (I) is that wherein R^{3a} is a ring of formula (a): 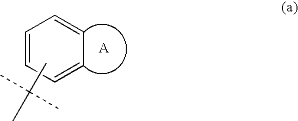
where A is a monocyclic five-, six-, or seven-membered heterocyclyl ring substituted with R⁴, R⁵ and R⁶, as defined in the Summary of the Invention.

(ii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, another group of compounds of Formula (I) is that wherein R^{3a} is a ring of formula: 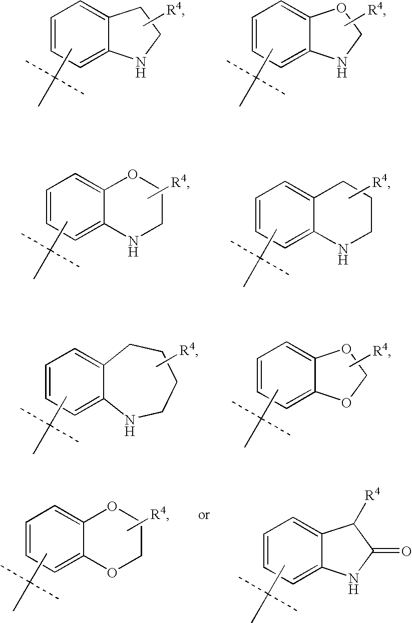
wherein R⁴ is as defined in the Summary of the invention.

(iii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, another group of compounds of Formula (I) is that wherein R^{3a} is a ring of formula: 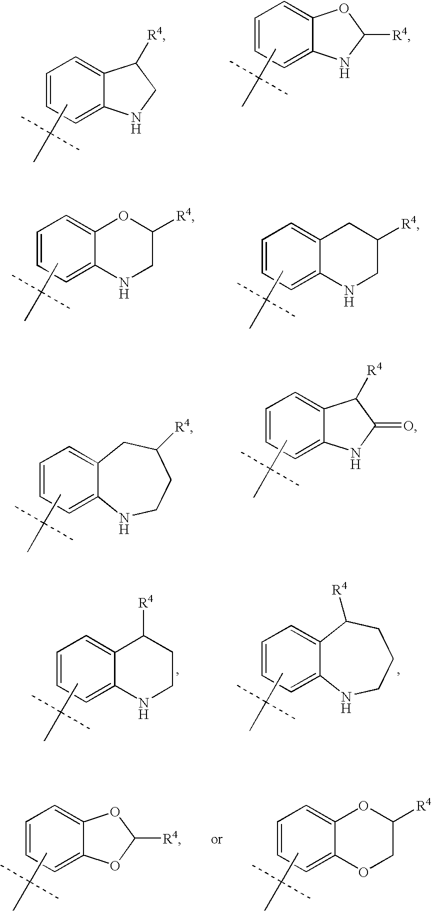
wherein R⁴ is as defined in the Summary of the invention.

(iv) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, another group of compounds of Formula (I) is that wherein R^{3a} is a ring of formula: 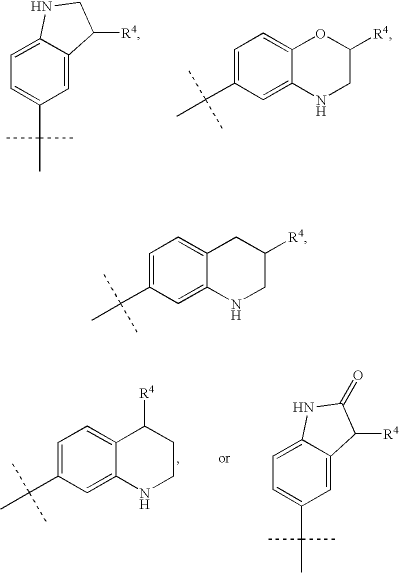
wherein R⁴ is as defined in the Summary of the invention.

Within the subgroups (ii)-(iv) above, one group of compounds is that wherein R⁴ is phenyl optionally substituted, as defined in the Summary of the Invention.

Within the subgroups (ii)-(iv) above, another group of compounds is that wherein R⁴ is heteroaryl optionally substituted, as defined in the Summary of the Invention.

Within the subgroups (ii)-(iv) above, yet another group of compounds is that wherein R⁴ is saturated monocyclic heterocyclyl optionally substituted, as defined in the Summary of the Invention.

Within the subgroups (ii)-(iv) above, another group of compounds is that wherein R⁴ is saturated fused heterocyclyl optionally substituted, as defined in the Summary of the Invention.

The R^{3a} rings in subgroups (ii)-(iv) above, the subgroups contained therein, including the hydrogen in —NH— groups in the rings, can also be optionally substituted with R⁵ and R⁶, where R⁵ and R⁶ are as defined in the Summary of the Invention. Preferably, one of R⁵ and R⁶is hydrogen.

(v) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 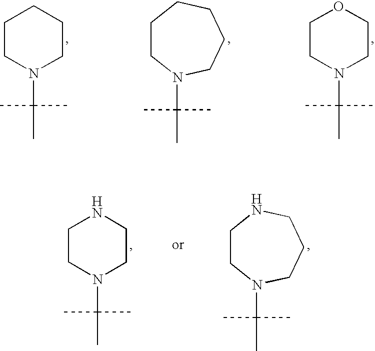
wherein the ring is substituted with R⁴, R⁵ and R⁶, as defined in the Summary of the Invention.

Within this subgroup, one group of compounds is that wherein the above rings are substituted with R⁴ as defined in the Summary of the Invention, and substituted with R⁵ and R⁶, where one of R⁵ and R⁶ is hydrogen. In one group of compounds, the —NH— groups in the rings are substituted with alkyl, cycloalkyl, or cycloalkylalkyl. In another group of compounds, the —NH— groups in the rings are unsubstituted. Within this embodiment, one group of compounds is that wherein R^{3a} is morpholin-4-yl, piperazin-1-yl, or homopiperazin-1-yl, substituted as defined above. Within this embodiment, another group of compounds is that wherein R^{3a} is piperidin-1-yl or homopiperidin-1-yl, substituted as defined above. Within this embodiment, another group of compounds is that wherein R^{3a} is morpholin-4-yl substituted as defined above.

(vi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 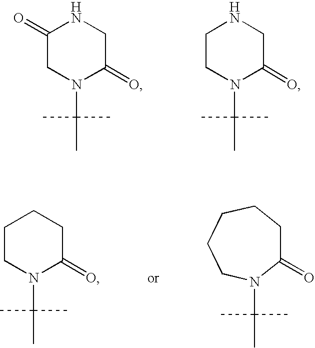
wherein the ring is substituted with R⁴, R⁵ and R⁶, as defined in the Summary of the Invention.

Within this subgroup, one group of compounds is that wherein the above rings are substituted with R⁴ as defined in the Summary of the Invention, preferably cycloalkyl, aryl, heteroaryl, or six-membered saturated heterocyclyl optionally substituted with R^{a}, R^{b} and R^{c}; and substituted with R⁵ and R⁶, where at least one of R⁵ and R⁶ is hydrogen. In one group of compounds, the —NH— groups in the rings are substituted with alkyl, cycloalkyl, or cycloalkylalkyl. In another group of compounds, the —NH— groups in the rings are unsubstituted.

(vii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 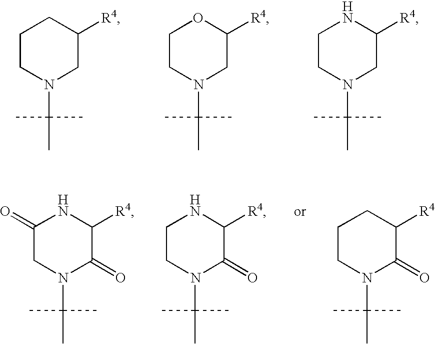
preferably, 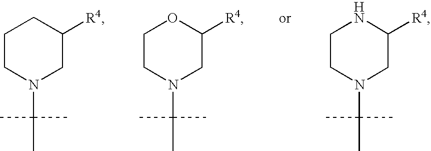
   where R⁴ is as defined in the Summary of the Invention.

Within this embodiment, one group of compounds is that wherein R⁴ is cycloalkyl, phenyl, heteroaryl, or six-membered saturated heterocyclyl, preferably cycloalkyl, aryl, heteroaryl, or six membered saturated heterocyclyl, optionally substituted with R^{a}, R^{b} and R^{c}. The rings of the formulas shown above are optionally substituted, including the hydrogen atom on the —NH— group within the rings, with R⁵ and R⁶, as defined in the Summary of the Invention; preferably, R⁵ is hydrogen ,and R⁶ is attached to the carbon adjacent to the nitrogen attached to the quinoline or isoquinoline ring. Within this embodiment, one group of compounds is that where R⁴ is phenyl substituted with R^{a} and R^{b} that are meta to each other.

Within this embodiment, one group of compounds is that wherein R^{3a} is morpholin-4-yl, piperazin-1-yl, 2-oxopiperidinyl, 2,4-dioxopiperazinyl, or 2-oxopiperazinyl, substituted as defined in (vi) above. Within this embodiment, another group of compounds is that wherein R^{3a} is piperidin-1-yl, substituted as defined in (vi) above. Within this embodiment, another group of compounds is that wherein R^{3a} is morpholin-4-yl substituted as defined in (vi) above.

(viii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 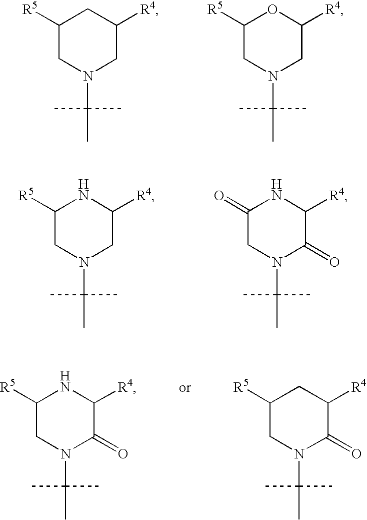
where R⁴ is phenyl or heteroaryl, substituted at the para position with R^{a}, and optionally substituted with R^{b} and R^{c}, wherein R^{a}, R^{b}, R^{c}, and R⁵ are as defined in the Summary of the Invention. The —NH— groups in the above rings can optionally be substitituted with R⁶ as defined in the Summary of the Invention. In one group of compounds within this embodiment, R⁶ is cycloalkyl, alkyl, or cycloalkylalkyl. In another group of compounds within this embodiment, R^{3a} is other than piperidin-1-yl substituted as described above. In another group of compounds within this embodiment, R^{3a} is piperidin-1-yl substituted as described above. In yet another group of compounds within this embodiment R^{3a} is morpholin-4-yl substituted as described above. In yet another group of compounds within this embodiment R^{3a} is morpholin-4-yl where R⁴ is phenyl is substituted with R^{a} and R^{b} where R^{a} and R^{b} are meta to each other. In yet another group of compounds within this embodiment R^{3a} is piperazin-1-yl where R⁴ is phenyl is substituted with R^{a} and R^{b} where R^{a} and R^{b} are meta to each other. In yet another group of compounds within this embodiment R⁴ is —CONR⁷R⁸ where R⁷ and R⁸ are as defined in the Summary of the Invention, preferably R⁷ is phenyl optionally substituted with R^{b} and R^{c} wherein R a, R^{b}, and R^{c} are as defined in the Summary of the Invention.

(ix) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 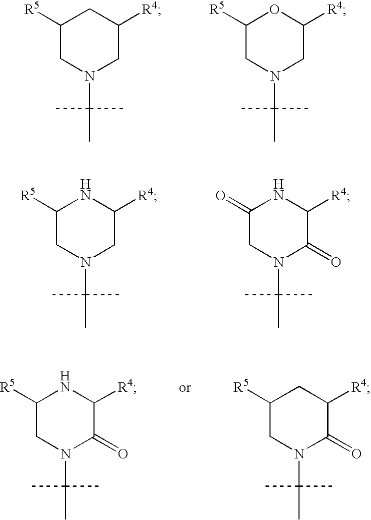
where R⁴ is heterocyclyl, preferably heterocyclyl containing at least a —C═O group wherein the heterocyclyl ring is optionally substituted at the para position with R^{a} and optionally substituted with R^{b} and R^{c} wherein R^{a}, R^{b}, and R^{c} are as defined in the Summary of the Invention and R⁵ is as defined in the Summary of the Invention. Within this group, in one embodiment, R⁴ is monocyclic saturated six membered ring containing at least a —C═O group and optionally substituted at the para position with R^{a} and optionally substituted with R^{b} and R^{c} wherein R^{a}, R^{b}, and R^{c} are as defined in the Summary of the Invention. The —NH— groups in the above rings can optionally be substitituted with R⁶ as defined in the Summary of the Invention. Preferably, R⁶ is cycloalkyl, alkyl, or cycloalkylalkyl. In one group of compounds within this embodiment R^{3a} is other than piperidin-1-yl substituted as described above. In one group of compounds within this embodiment, R^{3a} is piperidin-1-yl substituted as described above.

(x) Within the above embodiments (1)-(9), and embodiments contained therein i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 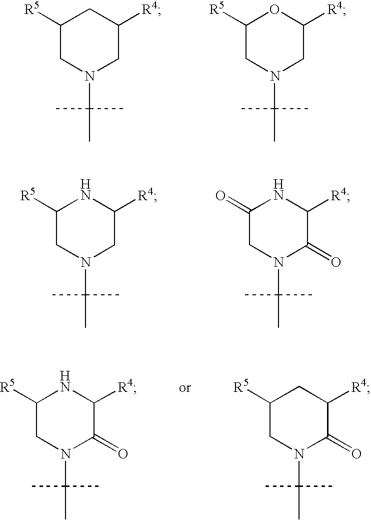
where R⁴ is cycloalkyl substituted at the para position with R^{a} and optionally substituted with R^{b} and R^{c} wherein R^{a}, R^{b} and R^{c} are as defined in the Summary of the Invention and R⁵ is as defined in the Summary of the Invention. The —NH— groups in the above rings can optionally be substitituted with R⁶ as defined in the Summary of the Invention. In one group of compounds within this embodiment R⁶ is cycloalkyl, alkyl, or cycloalkylalkyl.

(xi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 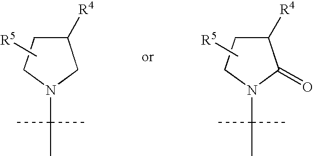
where R⁴ and R⁵ are as defined in the Summary of the Invention.

(xii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula (a) or (b), as defined in embodiments (xv) or (xvi), respectively. In one group of compounds is that wherein R^{3a} is a ring of formula: 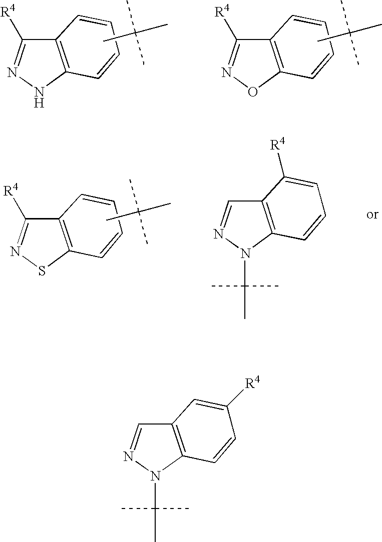
where R⁴ is cycloalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl). In certain subgroups, R⁴ is phenyl, heteroaryl or heterocyclyl. The rings shown in the formulas above are also optionally substituted, including the hydrogen in —NH— groups in the rings, with R⁵ and R⁶ where R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, or disubstituted amino. The aromatic or alicyclic ring in R⁴, R⁵, R⁶, and R⁷is optionally substituted with one to three substitutents independently selected from R^{a}, R^{b}, and R^{c} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, or disubstituted amino; and additionally substituted with one or two substitutents independently selected from R^{d} and R^{e} where R^{d} and R^{e} are hydrogen or fluoro.

Within this embodiment, one group of compounds is that wherein R^{3a} is: 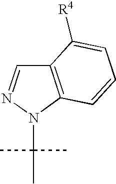
where R⁴ is phenyl, heteroaryl, or five- or six-membered heterocyclyl, each optionally substituted with one to three substitutents independently selected from R^{a}, R^{b}, and R^{c}, as defined in the Summary of the Invention.

Within this embodiment, another group of compounds is that wherein R^{3a} is: 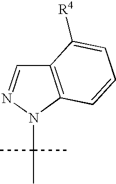
where R⁴ is morpholin-4-yl, piperazin-1-yl, or pyridinyl, each optionally substituted with one to three substitutents independently selected from R^{a}, R^{b}, and R^{c}, as defined in the Summary of the Invention.

Within this embodiment, one group of compounds is that wherein R^{3a} is: 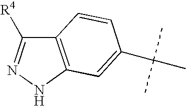
where R⁴ is phenyl, heteroaryl, or five- or six-membered heterocyclyl, each optionally substituted with one to three substitutents independently selected from R^{a}, R^{b}, and R^{c}, as defined in the Summary of the Invention and where the hydrogen in —NH— groups in the ring is optionally substituted with R⁵ and R⁶.

Within this embodiment, one group of compounds is that wherein R^{3a} is 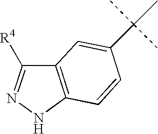
where R⁴ is phenyl, heteroaryl, or five- or six-membered heterocyclyl, each optionally substituted with one to three substitutents independently selected from R^{a}, R^{b}, and R^{c}, as defined in the Summary of the Invention and where the hydrogen in —NH— groups in the ring is optionally substituted with R⁵ and R⁶.

(xiii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 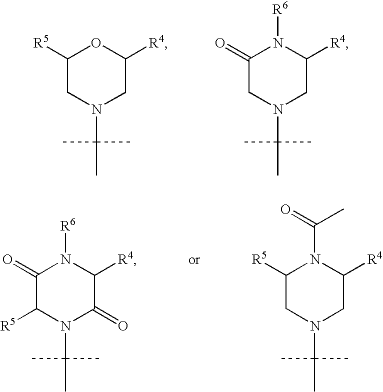
where R⁴ is cyclopentyl, cyclohexyl, phenyl, heteroaryl, or monocyclic saturated five- or six-membered heterocyclyl ring; R⁵ is hydrogen, alkyl, phenyl, heteroaryl, or monocyclic five- or six-membered heterocyclyl ring; and R⁶ is alkyl, preferably methyl; and wherein the aromatic or alicyclic ring in R⁴ and R⁵ is optionally substituted with R^{a}, R^{b} and R^{c}, as defined in the Summary of the Invention.

Within this subgroup, in one embodiment, R⁴ is phenyl, heteroaryl, or monocyclic five- or six-membered heterocyclyl ring and R⁵ is hydrogen or alkyl. In another embodiment, R⁴ and R⁵ are independently phenyl, heteroaryl, or monocyclic saturated five- or six-membered heterocyclyl ring. In each of the above embodiments, the aromatic or alicyclic ring is optionally substituted with R^{a} selected from alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, and optionally substituted heterocyclyl; and R^{b} and R^{c}, independently selected from alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, and disubstituted amino.

(xiv) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula: 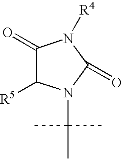
where R⁴ is aralkyl, preferably benzyl, optionally substituted with R^{a}, R^{b} and R^{c}, as defined in the Summary of the Invention; and R⁵ is as defined in the Summary of the Invention, preferably hydrogen or alkyl.

(xv) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula (a): 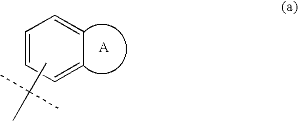
where A is a monocyclic five-, six-, or seven-membered heterocyclyl ring, and ring (a) is substituted with:

R⁴, where R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl, or heterocyclyl; and

R⁶, where R⁶ is hydrogen, alkyl, alkoxy, halo, haloalkyl, haloalkoxy, hydroxyl, cyano, carboxy, alkoxycarbonyl, alkylthio, sulfinyl, sulfonyl, acyl, aminocarbonyl, aminosulfinyl, aminosulfonyl, monosubstituted amino, or disubstituted amino, preferably hydrogen;

wherein the aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

In one embodiment, A is a saturated five or six membered heterocyclyl ring and substituted as described above.

(xvi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a ring of formula (b): 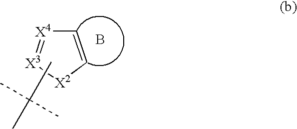
where:
X², X³, and X⁴ are independently carbon, nitrogen, oxygen, or sulfur; provided that at least two of X², X³, and X⁴ are other than carbon; and
B is phenyl, a six-membered heteroaryl ring (wherein the six-membered heteroaryl ring contains one or two nitrogen atoms, the rest of the ring atoms being carbon), or a monocyclic five-, six-, or seven-membered heterocyclyl ring;
wherein ring (b) is substituted with:

R⁴, where R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and

R⁶, where R⁶ is hydrogen, alkyl, alkoxy, halo, haloalkyl, haloalkoxy, hydroxyl, cyano, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen; and

wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro.

(xvii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a monocyclic six- or seven-membered heterocyclyl ring substituted with:

R⁴, where R⁴ is selected from cycloalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹¹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and

R⁵, where R⁵ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and

R⁶, where R⁶ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, or disubstituted amino, preferably hydrogen; and

wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro. In one group within this embodiment, R^{3a} is other than piperidinyl substituted as described above. In one group within this embodiment, R^{3a} is piperidinyl substituted as described above.

(xviii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is pyrrolidin-1-yl substituted with:

R⁴, where R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹¹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and

R⁶, where R⁶ is hydrogen, alkyl, alkoxy, halo, haloalkyl, haloalkoxy, hydroxyl, cyano, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen; and

wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro.

(xix) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is 2-oxopyrrolidinyl or 2,4-dioxoimidazolidinyl substituted with:
R⁴, where R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸-R¹²are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl);

R⁵, where R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and

R⁶, where R⁶ is hydrogen, alkyl, alkoxy, halo, haloalkyl, haloalkoxy, hydroxyl, cyano, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen; and

wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro.

(xx) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is phenyl optionally substituted as defined in the Summary of the Invention.

Within this embodiment, one group of compounds is that wherein R^{3a} is a group of formula: 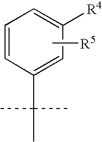
where one of R⁴ and R⁵ is hydrogen, alkyl, halo, haloalkyl, alkoxy, haloalkoxy, cyano, amino, monsubstituted or disubstituted amino, or —X¹R⁷ (where X¹ is —O—, —CO—, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is cycloalkyl, aryl, heteroaryl, or heterocyclyl; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

Preferably, R⁴ is aryl, heteroaryl, or heterocyclyl optionally substituted with one to three substitutents independently selected from R^{a}, R^{b}, and R^{c}.

Within this embodiment, one group of compounds is that wherein R^{3a} is a group of formula: 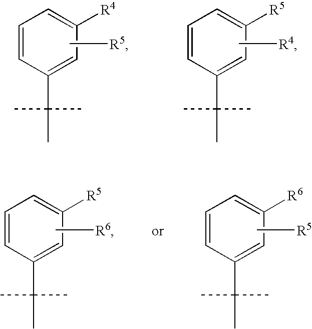
where R⁴ is hydrogen, alkyl, halo, haloalkyl, haloalkoxy or —X¹R⁷ (where X¹ is —O—, —CO—, —NR⁸CO—, —CONR⁹—, —S—, —SO—, —SO₂—, —NR¹¹SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); R⁵ is cycloalkyl, aryl, heteroaryl, or heterocyclyl; and R⁶ is alkoxy cyano, monsubstituted amino or disubstituted amino, wherein the aromatic or alicyclic ring in R⁵ and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

(xxi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 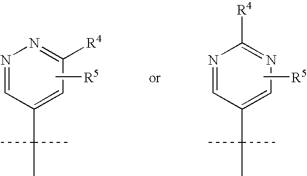
where R⁴ and R⁵ are as defined in (xvii) above.

(xxii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 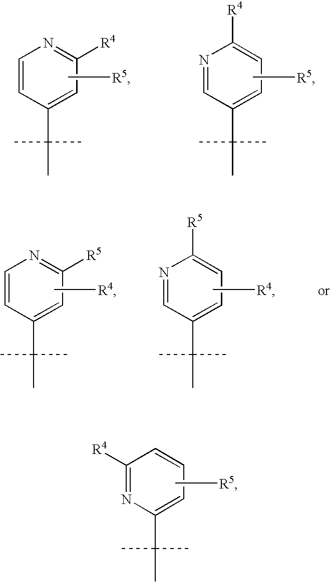
where R⁴ and R⁵ are as defined in (xxi) above.

One class of compounds is that where R^{3a} is a group of formula: 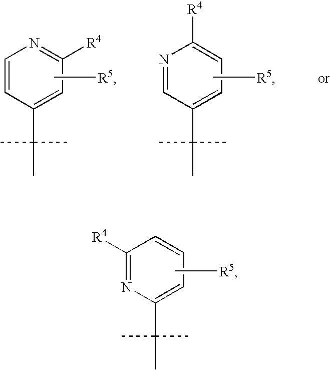
where R⁴ and R⁵ are as defined in (xxi) above.

Within this subgroup (xxii), another class of compounds is that where R⁴ is heteroaryl optionally substituted with one to three substitutents independently selected from R^{a}, R^{b}, and R^{c}.

Within this subgroup (xxii), another class of compounds is that where R⁴ is heterocyclyl, preferably piperazinyl, piperidinyl, or morpholinyl, each optionally substituted with one to three substitutents, independently selected from R^{a}, R^{b}, and R^{c}.

Within this subgroup (xxii), another class of compounds is that where R⁴ is mono or disubstituted amino and R⁵ is hydrogen, alkyl, or halo.

(xxiii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 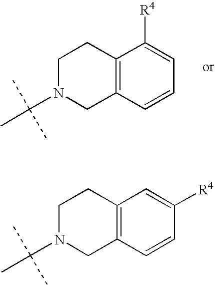
where R⁴ is as defined in the Summary of the Invention. The isoquinoline ring can optionally be substituted with R⁵ as defined in the Summary of the Invention.

Within this subgroup (xxiii), another class of compounds is that where R⁴ is heteroaryl optionally substituted with one to three substitutents independently selected from R^{a}, R^{b}, and R^{c}. Within this subgroup (xxiii), another class of compounds is that where R⁴ is heterocyclyl, preferably piperazinyl, piperidinyl, or morpholinyl, each optionally substituted with one to three substitutents, independently selected from R^{a}, R^{b}, and R^{c}.

(xxiv) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 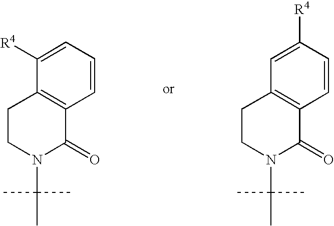
where R⁴ is as defined in the Summary of the Invention. The isoquinoline ring can optionally be substituted with R⁵ as defined in the Summary of the Invention.

Within this subgroup (xxiv), another class of compounds is that where R⁴ is heteroaryl optionally substituted with one to three substitutents independently selected from R^{a}, R^{b}, and R^{c}. Within this subgroup (xxiv), another class of compounds is that where R⁴ is heterocyclyl, preferably piperazinyl, piperidinyl, or morpholinyl, each optionally substituted with one to three substitutents, independently selected from R^{a}, R^{b}, and R^{c}.

(xxv) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: R⁴ R⁶ 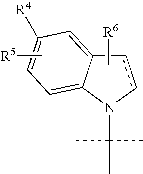
where “----” represents a single bond or a double bond, and R⁴, R⁵, and R⁶ are as defined in the Summary of the Invention.

Within this embodiment, one class of compounds is that wherein R^{3a} is a group of formula: 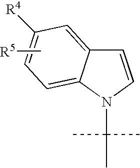
where one of R⁴ and R⁵ is hydrogen, alkyl, halo, haloalkyl, alkoxy, haloalkoxy, cyano, amino, monsubstituted or disubstituted amino, or —X¹R⁷ (where X¹ is —O—, —CO—, —NR⁸CO—, —CONR⁹—, —S—, —SO—, —SO₂—, —NR¹⁰SO₂—, or —SO₂NR¹¹— where R⁸-R¹¹ are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other one of R⁴ and R⁵ is cycloalkyl, aryl, heteroaryl, or heterocyclyl; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is aryl, heteroaryl, or heterocyclyl optionally substituted with one to three substitutents independently selected from R^{a}, R^{b}, and R^{c}.

Within this embodiment, another class of compounds is that wherein R^{3a} is a group of formula: 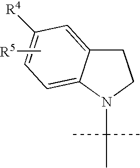
where R⁴ and R⁵ are as described immediately above.

(xxvi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another class of compounds is that wherein R^{3a} is a group of formula: 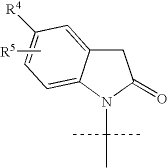
where R⁴ and R⁵ are as described in (xxv) above.

(xxvii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 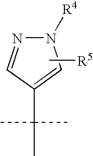
where R⁵ is hydrogen or alkyl, and R⁴ is aryl, heteroaryl, aralkyl, heteroaralkyl, or heterocyclyl, each optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

In one embodiment, R⁴ is aralkyl (preferably benzyl) optionally substituted with one to three substitutents independently selected from R^{a}, R^{b}, and R^{c}. In another embodiment, R⁴ is heteroaryl optionally substituted with one to three substitutents independently selected from R^{a}, R^{b}, and R^{c}. In one embodiment, R⁴ is heterocyclyl optionally substituted with optionally substituted phenyl or optionally substituted heteroaryl. In one class of compounds, R^{3a} is a group of formula: 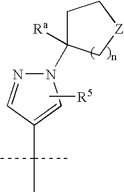
where R⁵ is hydrogen or alkyl, preferably hydrogen; n is 1, 2, or 3; Z is —O—, —NH—, or —N(alkyl)-; and R^{a} is phenyl or heteroaryl each optionally substituted with R^{a}, R^{b}, and R^{c}, preferably phenyl optionally substituted with R^{a}, R^{b}, and R^{c}.

(xxviii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 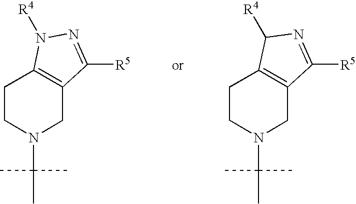
where R⁴ is hydrogen, alkyl, halo, haloalkyl, haloalkoxy, cycloalkyl, aryl, aralkyl, heteroaryl, heteroaralkyl, heterocyclyl, heterocyclylalkyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹ SO₂—, or —SO₂NR¹²- where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and R⁵ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl provided that at least one of R⁴and R⁵ is not hydrogen; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

Within this embodiment, one group of compounds is that wherein R⁴ is phenyl, heteroaryl, or heterocyclyl, each optionally substituted with one to three substitutents independently selected from R^{a}, R^{b}, and R^{c}.

(xxix) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 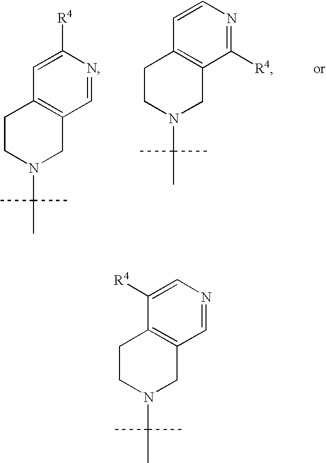
where R⁴ is alkyl, haloalkoxy, cycloalkyl, aryl, heteroaryl, heterocyclyl, or —X¹R⁷ (where X¹ is —O—, —CO—, —NR⁸CO—, —CONR⁹—, —NR¹⁰—, —S—, —SO—, —SO₂—, —NR¹¹ SO₂—, or —SO₂NR¹²— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl), wherein the aromatic or alicyclic ring in R⁴ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

Preferably, R⁴ is cycloalkyl, aryl, heteroaryl, or heterocyclyl, each optionally substituted with one to three substitutents independently selected from R^{a}, R^{b}, and R^{c}.

(xxx) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is a group of formula: 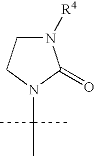
where R⁴ is aralkyl, preferably benzyl, optionally substituted with R^{a}, R^{b} and R^{c}, as defined in the Summary of the Invention.

(xxxi) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is pyridinyl, pyrimidinyl, benzoisothiazoly, or 5-, 6-, 7-, or 8-azaindolyl, each substituted as defined in the Summary of the Invention.

(xxxii) Within the above embodiments (1)-(9), and embodiments contained therein, i.e., (1)(A-P), (2)(A-P), (3)(A-P), (4)(A-P), (5)(A-P), (6)(A-P), (7)(A-P), (8)(A-P) and (9)(A-P), and groups contained therein, yet another group of compounds of Formula (I) is that wherein R^{3a} is morpholin-4-yl or piperazin-1-yl, each substituted as defined in the Summary of the Invention.

(xxxiii) Within the above embodiments (1)-(9), and embodiments contained 1)(A-P), (2)(A-P), (3)(A-P), (4)(A-P), (5)(A-P), (6)(A-P), (7)(A-P), (8)(A-P) and groups contained therein, yet another group of compounds of Formula (I) is R^{3a} is piperidinyl, piperazinyl, or morpholinyl, each optionally substituted as Summary of the Invention.

(xxxiv) Within the above embodiments (1)-(9), and embodiments contained 1)(A-P), (2)(A-P), (3)(A-P), (4)(A-P), (5)(A-P), (6)(A-P), (7)(A-P), (8)(A-P) and groups contained therein, yet another group of compounds of Formula (I) is R^{3a} is pyridinyl, pyrimidinyl, or isothiazolyl, each optionally substituted as Summary of the Invention.

Representative compounds of Formula (I) are provided in Table 1 below:

TABLE 1

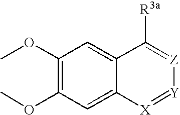

| Cpd # | X | Y | Z | R^{3a} |
|---|---|---|---|---|
| 1 | CH | CH | N | 2-(4-methoxyphenyl)morpholin-4-yl |
| 2 | CH | CH | N | 2-(morpholin-4-yl)pyridin-5-yl |
| 3 | N | CH | CH | 2-(4-methoxyphenyl)morpholin-4-yl |
| 4 | CH | CH | N | 3-(2-oxopiperidin-1-yl)piperidin-1-yl |
| 5 | N | CH | CH | 3-(2-oxopiperidin-1-yl)piperidin-1-yl |
| 6 | CH | CH | N | 2-(2S,6S-dimethylmorpholin-4-yl)pyridine-5-yl |
| 7 | CH | CH | N | 2-(4-methoxypiperidin-1-yl)pyridin-5-yl |
| 8 | CH | CH | N | 2-N-isopropylaminopyridin-5-yl |
| 9 | CH | CH | N | 2-N-ethyl-N-n-propylaminopyridin-5-yl |
| 10 | CH | N | CH | 2-methylbenzisotbiazol-5-yl |

### General Synthetic Schemes

Compounds of this invention can be made by the methods depicted in the reaction schemes shown below.

The starting materials and reagents used in preparing these compounds are either available from commercial suppliers, such as Aldrich Chemical Co. (Milwaukee, Wis.), Bachem (Torrance, Calif.), or Sigma (St. Louis, Mo.), or are prepared by methods known to those skilled in the art, following procedures set forth in references, such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including, but not limited to, filtration, distillation, crystallization, chromatography, and the like. Such materials may be characterized using conventional means, including physical constants and spectral data.

Unless specified to the contrary, the reactions described herein take place at atmospheric pressure over a temperature range from about −78° C. to about 150° C., from about 0° C. to about 125° C., or at about room (or ambient) temperature, e.g., about 23° C.

Compounds of Formula (I), where Z is nitrogen; and X is —CR═ (where R is hydrogen, alkyl, alkoxy, or halo), Y is carbon, and R¹, R², R³, and R^{3a} are as defined in the Summary of the Invention, can be prepared as described in Scheme 1.

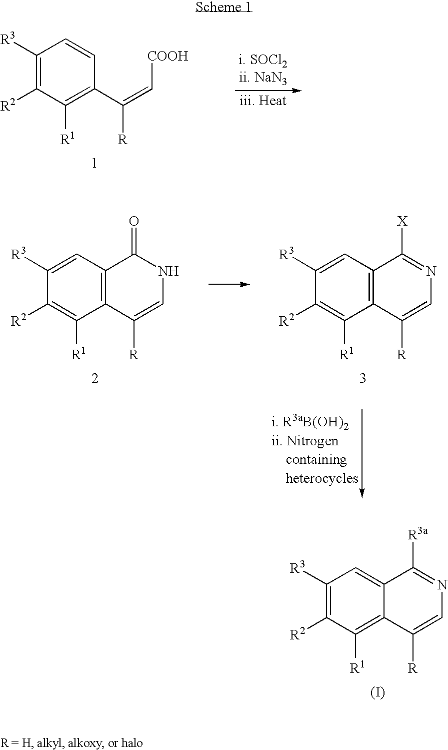

Treatment of acrylic acid derivative 1 with a halogenating agent, such as thionyl chloride, followed by treatment with sodium azide, provides acryloyl azide, which upon heating at approximately 270° C. in a suitable high boiling solvent, such as diphenylether, cyclizes to form the corresponding 2H-isoquinolin-1-one 2. Compounds of formula 1 are either commercially available or can be synthesized by methods common to the art.

Compound 2, where R is alkyl or halo, such as chloro or bromo, can be prepared by treating compound 2, where R is H, with a halogenating agent, such as N-chlorosuccinimide or N-bromosuccinimide, in N,N-dimethylformamide (see, Journal of Heterocyclic Chemistry, 38:597-600, 2001). Treatment of the resulting halo compound 2 with an alkyl Grignard reagent provides compound 2, where R is alkyl. 2H-Isoquinolin-1-one 2 is then converted to compound 3, where X is chloro or bromo, by treatment with phosphorus oxychloride or phosphorous oxybromide, respectively.

Compound 3 is converted into the corresponding compound of Formula (I) via a variety of methods. For example, compounds of Formula (I), wherein R^{3a} is an aryl or heteroaryl ring, can be prepared by standard synthetic methods known to one of ordinary skill in the art, e.g., Suzuki-type coupling of the corresponding aryl or heteroaryl boronic acid with compound 3 where X is halo (see, Miyaura and Suzuki, Chem. Rev., 95:2457-2483, 1995). Such boronic acids are either commercially available, e.g., Aldrich Chemical Co. (Milwaukee, Wis.), Lancaster Synthesis (Ward Hill, Mass.), or Maybridge (Cornwall, UK), or can readily be prepared from the corresponding bromides by methods described in the literature (see, Miyaura et al, Tetrahedron Letters, 1979, 3437; Miyaura and Suzuki, Chem. Commun. 1979, 866).

Compounds of Formula (I), where R^{3a} is a heterocyclic ring (e.g., pyrrolidin-1-yl, piperidin-1-yl, or morpolin-4-yl) attached via a nitrogen atom, can be prepared by reacting compound 3 with a heterocyclic ring in the presence of a base, such as triethylamine or pyridine. Suitable solvents include, but are not limited to, polar aprotic solvents, such as tetrahydrofuran and N,N-dimethylforamide (DMF). Such heterocyclic rings (pyrrolidines, piperidines, homopiperidines, piperazines, homopiperazines, morpholines, and the like) are either commercially available, or can be readily prepared by standard methods known within the art (see, Louie and Hartwig, Tetrahedron Letters, 36:3609, 1995; Guram et al., Angew Chem. Int. Ed., 34:1348, 1995).

Alternatively, a compound of Formula (I) can be prepared by heating compound 3 with a heterocyclic ring in a suitable organic solvent, such as tetrahydrofuran (THF), benzene, dioxane, toluene, alcohol, or a mixture thereof, under catalytic conditions, using, for example, a palladium or copper catalyst, such as, but not limited to, tris(dibenzylidene-acetone) dipalladium(0) or copper (I) iodide, in the presence of a suitable base, such as potassium carbonate, sodium t-butoxide, lithium hexamethyldisilizane, and the like.

Substituted indazoles useful to make compounds of Formula (I) are either commercially available, e.g., Aldrich Chemical Co. (Milwaukee, Wis.), Sinova, Inc. (Bethesda, Md.), J & W PharmLab, LLC (Morrisville, Pa.), or can be prepared by methods commonly known within the art (see, Lebedev et al, J. Org. Chem. 70:596-602, 2005; and the references cited therein). For example, indazoles wherein R⁴ is heterocyclyl, e.g., morpholine or N-methylpiperazine, may be synthesized by Buchwald-type coupling of the corresponding bromoindazole with the desired heterocyclic compound. The bromoindazoles may be prepared as described in International Publication No. WO 2004/029050, the disclosure of which is incorporated herein by reference in its entirety. Copper catalyzed reaction of the appropriately substituted indazole with compound 3, where X is halo, provides the desired compounds of Formula (I). Alternatively, the bromoindazole undergoes palladium catalyzed reaction with compound 3 (X is halo) to provide a 4-(bromo-1H-indazol-1-yl) substituted compound of Formula (I). Subsequent N-arylation reaction with, for example, morpholine or N-methylpiperazine, provides a desired compound of Formula (I). Alternatively, Suzuki-type reaction of the 4-(bromo- 1H-indazol-1-yl)-substituted compound with an aryl or heteroaryl boronic acid (e.g., phenylboronic acid or 4-pyridine boronic acid) gives the corresponding 4-aryl or heteroaryl substituted indazole compound of Formula (I).

Compounds of Formula (I), where Y is nitrogen, X is —CR═ (R=alkyl), Z is carbon, and R¹, R², R³ and R^{3a} are as defined in the Summary of the Invention, can be prepared as described in Scheme 2 below.

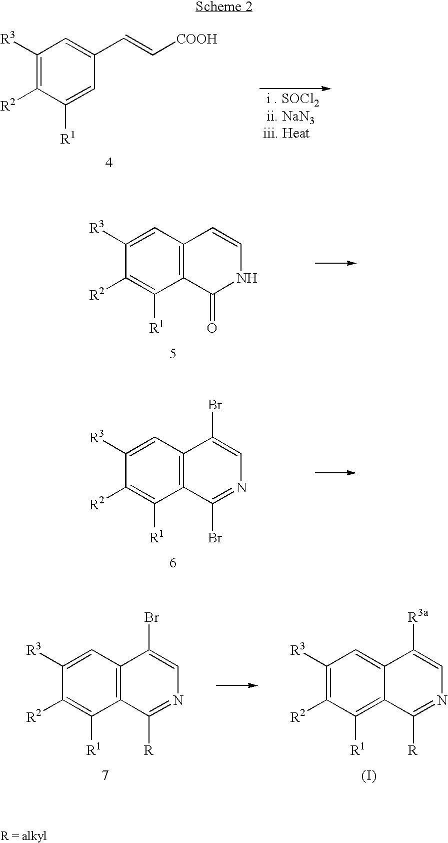

An acrylic acid derivative 4 is converted to the corresponding 2H-isoquinolin-1-one 5 under reaction conditions described in Scheme 1. Treatment of isoquinolin-1-one 5 with N-bromosuccinimide in N,B-dimethylformamide (see, Journal of Heterocyclic Chemistry, 38:597-600, 2001), followed by phosphorus oxybromide, provides 1,4-dibromoisoquinoline 6. Treatment of compound 6 with a suitable Grignard reagent, catalyzed by a palladium or copper catalyst, provides 1-alkyl-4-bromo-isoquinoline 7. Compounds of formula 4 are either commercially available or can be synthesized by methods common to the art. Alternatively, compound 5 can be converted to the corresponding 1,4-dichloroisoquinoline derivative by treating it with phosphorus pentachloride at elevated temperatures (see, Barber et al., Bioorg. Med. Chem. Lett., 14:3227-3230, 2004). Compound 7 is then converted to a compound of Formula (I) as described in Scheme I above.

Compounds of Formula (I), where X is nitrogen, Y and Z are —CH═, and R¹, R², R³ and R^{3a} are as defined in the Summary of the Invention, can be prepared as described in Scheme 3 below (see, J. Med. Chem., 42:5369, 1999).

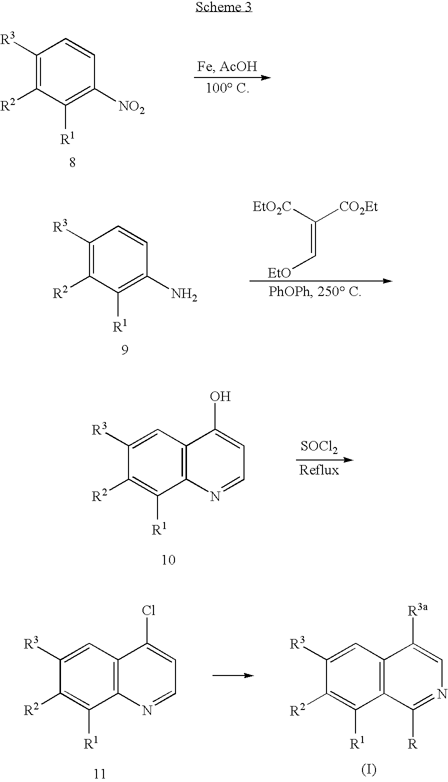

Compounds 9, where R¹ is hydrogen, and R² and R³ are the same and are selected from alkoxy, haloalkoxy, hydroxy, cycloalkyloxy, cycloalkylalkyloxy, hydroxyalkyloxy, alkoxyalkyloxy, or —O-(alkylene)-NR¹⁵R¹⁶, for example, methoxy, can be synthesized by methods common to the art. For example, 3,4-dihydroxy-nitrobenzene 8 (R¹=H, R²=R³=OH) can be treated with a desired R³LG, where R³ is as defined above and LG is a suitable leaving group, in the presence of a base, such as cesium carbonate, triethylamine, sodium hydride, potassium carbonate, potassium hydride, or the like, to provide the corresponding dialkoxy product. Suitable organic solvents include acetone, acetonitrile, DMF, THF, and the like. Reduction of the nitro group under known reaction conditions, e.g., hydrogenation with palladium on carbon, iron powder in acetic acid, or nickel boride, provides the amino compound 9 (see, Castle et al. J. Org. Chem. 19:1117, 1954).

Compounds 9, where R¹ is hydrogen, R² is haloalkoxy, hydroxy, cycloalkyloxy, cycloalkylalkyloxy, hydroxyalkyloxy, alkoxyalkyloxy, or —O—(alkylene)-NR¹⁵R¹⁶, and R³ is methoxy, can be prepared from 2-methoxy-5-nitrophenol as a starting material. Simple etherification, as described above, can be utilized to provide the required R² substitution, which, when followed by the reduction step as described above, provides the desired amino compound 9. Treatment of intermediate phenols with haloacetic acid, e.g., chlorodifluoroacetic acid, under basic conditions provides difluoromethyl ethers. Heating compound 9 with diethyl 2-(ethoxymethylene)malonate in the presence of diphenylether provides 4-hydroxyquinoline 10, which is then converted to 4-halo compound 11. Compound 11 is converted to a compound of Formula (I) as described in Scheme 1 above.

4-Chloroquinoline 11, where R¹ is hydrogen, and R² and R³ are halo, can be prepared as shown Scheme 4 below, which exemplifies the synthesis of 4-chloro-6,7-difluoroquinoline 16 (see, Bioorg. Med Chem., 13:2021, 2005; and PCT Application Publication No. WO 95/23787).

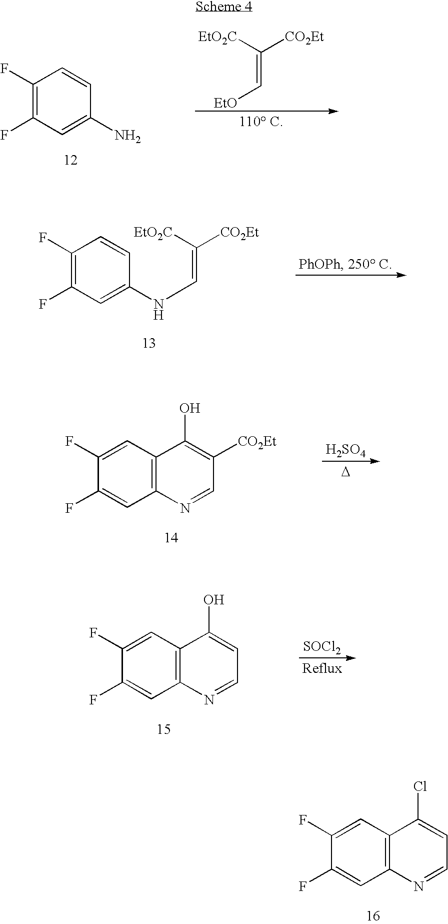

### Utility and Methods of Use

Provided herein are methods for treating a disorder or disease by inhibiting PDE10 enzyme. The methods, in general, comprises the step of administering a therapeutically effective amount of a compound of Formula (I), or an individual stereoisomer, a mixture of stereoisomers, or a pharmaceutically acceptable salt or solvate thereof, to a patient in need thereof to treat the disorder or disease.

The compounds of the present invention inhibit PDE10 enzyme activity, and hence raise the levels of cAMP or cGMP within cells that express PDE10. Accordingly, inhibition of PDE10 enzyme activity would be useful in the treatment of diseases caused by deficient amounts of cAMP or cGMP in cells. PDE10 inhibitors would also be of benefit in cases wherein raising the amount of cAMP or cGMP above normal levels results in a therapeutic effect. Inhibitors of PDE10 may be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex, and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention are suitable for use in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways (Saxena et al., Br. J Psychiatry Suppl, 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention are therefore suitable for use in the indication of OCD. OCD may result, in some cases, from streptococcal infections that cause autoimmune reactions in the basal ganglia (Giedd et al., Am J Psychiatry. 157:281-283, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that, since PDE10 degrades cAMP or cGMP, the level of this enzyme affects memory in animals, for example, in humans. A compound that inhibits cAMP phosphodiesterase (PDE) can thereby increase intracellular levels of cAMP, which in turn activate a protein kinase that phosphorylates a transcription factor (cAMP response binding protein). The phosphoylated transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention are suitable for use in treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention are suitable for use in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-I (ataxin-1); spinocerebellar ataxia type-2 (ataxin-2); spinocerebellar ataxia type-3 (also called Machado-Joseph disease or MJD) (ataxin-3); spinocerebellar ataxia type-6 (alpha la-voltage dependent calcium channel); spinocerebellar ataxia type-7 (ataxin-7); and spinal and bulbar muscular atrophy (SBMA, also know as Kennedy disease).

The basal ganglia are important for regulating the function of motor neurons; disorders of the basal ganglia result in movement disorders. Most prominent among the movement disorders related to basal ganglia function is Parkinson's disease (Obeso et al., Neurology. 62(1 Suppl 1):S17-30, 2004). Other movement disorders related to dysfunction of the basal ganglia include tardive dyskinesia, progressive supranuclear palsy and cerebral palsy, corticobasal degeneration, multiple system atrophy, Wilson disease, dystonia, tics, and chorea. The compounds of the invention are also suitable for use to treat movement disorders related to dysfunction of basal ganglia neurons.

PDE10 inhibitors are useful in raising cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis.

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity inhibits cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention can be used to stop the growth of cancer cells that express PDE10.

The compounds of the invention are also suitable for use in the treatment of diabetes and related disorders such as obesity, by focusing on regulation of the cAMP signaling system. By inhibiting PDE-10, especially PDE-10A, intracellular levels of cAMP are increased, thereby increasing the release of insulin-containing secretory granules and, therefore, increasing insulin secretion. See, for example, WO 2005/012485, which is hereby incorporated by reference in its entirety. The compounds of Formula (I) can also be used to treat diseases disclosed in US Patent application publication No. 2006/019975, the disclosure of which is incorporated herein by reference in its entirety.

### Testing

The PDE10 inhibitory activities of the compounds of the present invention can be tested, for example, using the in vitro and in vivo assays described in the Biological Examples below.

### Administration and Pharmaceutical Compositions

In general, the compounds of this invention can be administered in a therapeutically effective amount by any of the accepted modes of administration for agents that serve similar utilities. The actual amount of a compound of this invention, i.e., the active ingredient, depends upon numerous factors, such as the severity of the disease to be treated, the age and relative health of the subject, the potency of the compound used, the route and form of administration, and other factors.

Therapeutically effective amounts of compounds of formula (I) may range from approximately 0.1-1000 mg per day; preferably 0.5 to 250 mg/day, more preferably 3.5 mg to 70 mg per day.

In general, compounds of this invention can be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors, such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, Gennaro, A. R. (Mack Publishing Company, 18th ed., 1995).

The level of the compound in a formulation can vary within the full range employed by those skilled in the art. Typically, the formulation contains, on a weight percent (wt %) basis, from about 0.01-99.99 wt % of a compound of Formula (I) based on the total formulation, with the balance being one or more suitable pharmaceutical excipients. Preferably, the compound is present at a level of about 1-80 wt %. with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic a-7 agonists, PDE4 inhibitors, other PDE10 inhibitors, calcium channel blockers, muscarinic ml and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range, and can be administered either simultaneously or sequentially.

Drugs suitable in combination with the compounds of the present invention include, but are not limited to, other suitable schizophrenia drugs such as Clozaril, Zyprexa, Risperidone, and Seroquel; bipolar disorder drugs, including, but not limited to, Lithium, Zyprexa, and Depakote; Parkinson's disease drugs, including, but not limited to, Levodopa, Parlodel, Permax, Mirapex, Tasmar, Contan, Kemadin, Artane, and Cogentin; agents used in the treatment of Alzheimer's disease, including, but not limited to, Reminyl, Cognex, Aricept, Exelon, Akatinol, Neotropin, Eldepryl, Estrogen and Cliquinol; agents used in the treatment of dementia, including, but not limited to, Thioridazine, Haloperidol, Risperidone, Cognex, Aricept, and Exelon; agents used in the treatment of epilepsy, including, but not limited to, Dilantin, Luminol, Tegretol, Depakote, Depakene, Zarontin, Neurontin, Barbita, Solfeton, and Felbatol; agents used in the treatment of multiple sclerosis, including, but not limited to, Detrol, Ditropan XL, OxyContin, Betaseron, Avonex, Azothioprine, Methotrexate, and Copaxone; agents used in the treatment of Huntington's disease, including, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (e.g., sulfonylurea drugs, such as Glyburide, Glimepiride, Chlorpropamide, Tolbutamide, and Glipizide, and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors, and 11beta-HSD inhibitors), hepatic glucose output lowering compounds (such as glucagon antagonists and metaformin, e.g., Glucophage and Glucophage XR), insulin and insulin derivatives (both long and short acting forms and formulations of insulin); and anti-obesity drugs, including, but not limited to, β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat).

### EXAMPLES

The following preparations and examples are given to enable those skilled in the art to more clearly understand and to practice the present invention. They should not be considered as limiting the scope of the invention, but merely as being illustrative and representative thereof.

All NMR spectra were recorded at 300 MHz on a Bruker Instruments NMR unless otherwise stated. Coupling constants (J) are in Hertz (Hz) and peaks are listed relative to TMS (δ 0.00 ppm). Microwave reactions were performed using a Personal Chemistry Optimizer™ microwave reactor in Personal Chemistry microwave reactor vials. Sulfonic acid ion exchange resins (SCX) were purchased from Varian Technologies. Analytical HPLC was performed on 4.6 mm×100 mm Waters Sunfire RP C18 5 μm column. 4-Bromo-6,7-dimethoxyquinoline, a starting material for making certain compounds of Formula (I), is commercially available.

### Synthetic Examples

### Example 1

### Synthesis of 1-bromo-6,7-dimethoxyisoquinoline

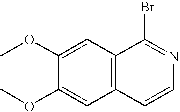

Step 1. A mixture of 3,4-dimethoxybenzaldehyde (30 g, 180.72 mmol), malonic acid (28.4 g, 273.08 mmol), and piperidine (3 mL) in pyridine (90 mL) was stirred at 120° C. for 6 hr. The reaction mixture was monitored by TLC (EtOAc/PE (1:1, v/v)). Upon completion, the reaction mixture was cooled to room temperature, and the pH was then adjusted to 1 by the addition of concentrated HCl. The product was isolated by filtration, and the filter cake was washed with water. The solid was dried in an oven under reduced pressure to provide 30 g (80%) of (E)-3-(3,4-dimethoxyphenyl)acrylic acid as a light yellow solid.

Step 2. To a solution of (E)-3-(3,4-dimethoxyphenyl) acrylic acid (10 g, 48.08 mmol) in THF (500 mL) was added a solution of DPPA (13.3 g, 48.36 mmol) in THF (20 mL) dropwise with stirring at 0 to 5° C. TEA (5 g, 49.50 mmol) was then added dropwise with stirring over a time period of 1.5 hr, and the resulting mixture was stirred for additional 12 hr at room temperature. The reaction mixture was concentrated, followed by the dropwise addition of CH₃OH (300 mL) with stirring. The resulting solution was refluxed for additional 48 hr. The reaction was monitored by TLC (EtOAc:PE(1:1, v/v). The reaction mixture was quenched by the addition of H₂O, and then extracted with EtOAc and the organic layers combined. The residue was purified by silica gel chromatography using EtOAc/PE (1:10, v/v) as an eluant to provide (E)-methyl 3,4-dimethoxystyrylcarbamate as a white solid (2.5 g).

Step 3. A solution of (E)-methyl-3,4-dimethoxystyrylcarbamate (15 g, 63.29 mmol) and Bu₃N (7.5 g) in 1-phenoxybenzene (150 mL) was refluxed for 12 hr. The reaction was monitored by TLC (EtOAc/PE (1:1. v/v)). Upon completion, PE (2 L) was added, and the product was isolated by filtration to provide 6,7-dimethoxy-isoquinolin-1(2H)-one as a light yellow solid (2.0 g).

Step 4. A solution of 6,7-dimethoxyisoquinolin-1(2H)-one (2 g, 8.29 mmol) and phosphorus oxybromide (14 g, 48.78 mmol) in dry acetonitrile (200 mL) was reflexed for 4 hr. The reaction mixture was monitored by TLC(EtOAc:PE (1:1, v/v)). Upon completion, the reaction was quenched with ice. The reaction mixture was neutralized with solid potassium carbonate. The resulting aqueous solution was extracted three times with ethyl acetate. The combined organic layers were washed with water and saturated sodium chloride solution, dried with anhydrous magnesium sulfate, filtered, and concentrated. The crude product was purified by silica gel chromatography using EtOAc/hexane (1:10, v/v) as an eluant to provide 1-bromo-6,7-dimethoxyisoquinoline as a yellow solid (650 mg, 51%). ¹H NMR (400 MHz, DMSO) δ: 3.99 (6H, s), 7.41(1H, s), 7.49 (H, s), 7.75 (1H, d), 8.13 (1H, d). LCMS [M+H]⁺ calcd for C₁₁H₁₁BrNO 269, found 269.

### Example 2

### Synthesis of 6,7-dimethoxy-1-methyl-isoquinolin-4-yl-trifluoromethanesulfonate

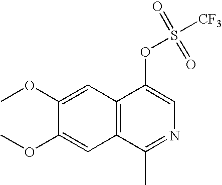

Step 1. Acetic anhydride (150 mL) was added to a mixture of 2-(3,4-dimethoxyphenyl)ethanamine (40 g, 220.99 mmol), DMAP (2 g, 16.39 mmol), and Et₃N (40 g, 396.04 mmol) in a 500 mL 3-necked round bottom flask. The resulting solution was stirred for 5 hr at room temperature. The reaction was monitored by TLC (EtOAc:PE, (1:1, v/v)). A filtration was performed to provide N-(3,4-dimethoxy-phenethyl)acetamide as a white solid (32 g).

Step 2. A mixture of N-(3,4-dimethoxyphenethyl)acetamide (25 g, 112.11 mmol) and POCl₃ (37 mL) in toluene (187 mL) was stirred at 120° C. for 3.5 hr. The reaction was monitored by TLC (EtOAc:PE (1:1, v/v)). Upon completion, the reaction mixture was cooled to room temperature and the pH was adjusted to 12 by the addition of NaOH (4N). The resulting mixture was washed with EtOAc and filtration was performed to yield 6,7-dimethoxy-1-methyl-3,4-dihydroisoquinoline as a yellow solid (20 g).

Step 3. Into a 1000 mL 3-necked round bottom flask purged and maintained with an inert atmosphere of nitrogen while cooling in an ice bath at 0° C. was added 6,7-dimethoxy-1-methyl-3,4-dihydroisoquinoline (8 g, 39.02 mmol), 1,2,3,4-tetrahydronaphthalene (650 mL) and Pd/C (8 g). The reaction mixture was then refluxed for 3 hr. The reaction was monitored by TLC (EtOAc/MeOH (10:1, v/v)). Upon completion, the reaction mixture was cooled to room temperature, and filtered. The pH was adjusted to 2 by the addition of 10% aqueous HCl. The aqueous layer was separated and the pH was adjusted to 10 by the addition of 10% aqueous NaOH. The resulting solution was extracted with EtOAc, and the organic fraction was dried over anhydrous Na₂SO₄ and concentrated to provide 6,7-dimethoxy-1-methylisoquinoline as a brown solid (7 g).

Step 4. A solution of 6,7-dimethoxy-1-methylisoquinoline (3.3 g, 16.26 mmol) and m-CPBA (3.7 g, 21.45 mmol) in DCM (80 mL) was refluxed overnight. The mixture was cooled to room temperature, the pH was adjusted to 8 by the addition of NaOH (4N), and then extracted one time with EtOAc. The organic fraction was dried over anhydrous Na₂SO₄ and concentrated to provide 6,7-dimethoxy-1-methyl-isoquinoline-N-oxide as a yellow solid (3.3 g).

Step 5. A solution of 6,7-dimethoxy-1-methylisoquinoline-N-oxide (500 mg, 2.46 mmol) and NaOAc (0.6 g) in Ac₂O (5 mL) and AcOH (3 mL) was stirred at 85° C. for 2 hr and the reaction was monitored by TLC (DCM:MeOH (10:1, v/v)). Upon completion, the reaction mixture was concentrated, taken up in 50 mL of H₂O and 100 mL of CH₂Cl₂. The organic fraction was separated, washed with Na₂CO₃, dried over anydrous Na₂SO₄ and concentrated. The mixture was diluted with 20 mL of 10% HCl, refluxed for 1 hr. Upon cooling to room temperature, the pH was adjusted to 7 by the addition of aqueous Na₂CO₃, and then extracted with CH₂Cl₂. The organic fraction was dried over anydrous Na₂SO₄, concentrated and purified by silica gel chromatography using a gradient elution going from 15:1 (v/v) to 10:1 (v/v) of DCM:MeOH to provide 6,7-dimethoxy-1-methylisoquinolin-4-ol as a brown solid (20 mg).

Step 6. To a solution of 6,7-dimethoxy-1-methylisoquinolin-4-ol (400 mg, 1.83 mmol) in DCM (30 mL) in the presence of Et₃N (930 mg, 9.21 mmol) was added Tf₂O (780 mg, 2.77 mmol) dropwisely with stirring at 0° C., and the reaction mixture was stirred for 20 min at 0° C. The reaction was monitored by TLC (CH₃OH: CH₂Cl₂ (1:10, v/v)). Upon completion, the resulting mixture was washed with H₂O, dried over anhydrous Na₂SO₄, and concentrated. The residue was purified by silica gel chromatography using 1:6 (v/v) EtOAc/PE as an eluant to provide 6,7-dimethoxy-1-methylisoquinolin-4-yl trifluoromethanesulfonate as a white solid (265 mg). ¹H NMR (400 Hz, DMSO) δ 2.88(3H,S), 3.98(3H,S), 4.01(3H,S), 7.19(1H,S), 7.52(1H,S), 8.37(1H,S). LCMS [M+H]⁺ calcd for C₁₃H₁₃F₃NO₅S 352, found 352.

### Example 3

### Synthesis of 6,7-dimethoxyisoquinolin-4-yl-trifluoromethanesulfonate

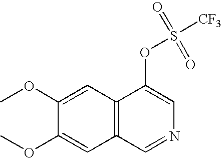

Step 1. To a solution of ethyl 2-aminoacetate hydrochloride (20 g, 143.37 mmol) in MeOH (300 mL) was added Et₃N (14.6 g, 144.27 mmol) dropwise at 0° C. The reaction mixture was stirred for 10-20 min and then 3,4-dimethoxybenzaldehyde (24 g) was added in several batches. The resulting solution was stirred for 2 hr, and then NaBH₄ (11 g, 297.30 mmol) was added in several batches. The resulting solution was stirred overnight at room temperature. The reaction was monitored by TLC (EtOAc/PE, (1:2, v/v)). The reaction mixture was concentrated, quenched by adding H₂O and extracted with several portions of EtOAc. The combined organic layers were dried over anhydrous Na₂SO₄, and concentrated to provide ethyl 2-(3,4-dimethoxybenzyl-amino)acetate as a yellow solid (37 g).

Alternatively, ethyl 2-(3,4-dimethoxybenzylamino)acetate was also prepared as following. To a stirred solution of 3,4-dimethoxy benzaldehyde (25 g, 150.5 mmol) in dichloroethane (250 ml) was added glycine ethyl ester (25.2 g, 180.6 mmol) and magnesium sulfate (40 g). Triethyl amine (42.23 ml, 301 mmol) was then added dropwise at 0° C. over 60 min. The resulting solution was first brought to room temperature and then stirred for overnight. Sodium triacetoxy borohydride (64 g. 301 mmol) was added in portions at 0° C. and the reaction mixture was stirred at room temperature overnight. The reaction was monitored by TLC. The reaction mixture was filtered out and washed with DCM (200 ml). The filtrate was concentrated, and the residue was dissolved in H₂O and the resulting mixture was washed with ethyl acetate to remove non-polar impurities. The mixture was adjusted to pH 8 with NaHCO₃ and then extracted with ethyl acetate. The combined organic layers were dried over anhydrous Na₂SO₄ and concentrated to give ethyl 2-(3,4-dimethoxybenzylamino)acetate as an oil (21 g, 56%).

Step 2. To a solution of ethyl 2-(3,4-dimethoxybenzylamino) acetate (50 g, 167.98 mmol) and DMAP (1.2 g, 9.68 mmol) in DCM (300 mL) in the presence of Et₃N (52 g, 514.85 mmol) was added 4-methylbenzene-1-sulfonyl chloride (41 g, 215.79 mmol) dropwise with stirring at 0° C. The resulting solution was stirred for 1 hr at room temperature and then quenched by adding 2N HCl. The reaction solution was washed with H₂O and brine, dried over anydrous Na₂SO₄, and concentrated. The crude product was purified by silica gel chromatography using 1:5 (v/v) EtOAc:PE as an eluant to provide ethyl 2-(N-(3,4-dimethoxybenzyl)-4-methylphenylsulfonamido)acetate as a white solid (55 g).

Step 3. A solution of ethyl 2-(N-(3,4-dimethoxybenzyl)-4-methylphenylsulfonamido)acetate (55 g, 135.14 mmol) in 15% NaOH (300 mL) was refluxed for 30 min. The reaction mixture was cooled in a bath of H₂O/ice, and the pH was adjusted to 5-6 with 2N HCl solution. The resulting mixture was extracted with EtOAc three times, and the organic layers were combined, washed with water and brine, dried over anhydrous Na₂SO₄, and concentrated to provide 2-(N-(3,4-dimethoxybenzyl)-4-methylphenylsulfonamido)acetic acid as a white solid (47 g) which was used in the next step without further purification.

Step 4. To a solution of 2-(N-(3,4-dimethoxybenzyl)-4-methylphenyl-sulfonamido)acetic acid (47 g, 124.01 mmol) in dichloromethane (300 mL) was added oxalyl chloride (78 g, 655.46 mmol) at 0° C. The resulting solution was refluxed for 5 hr. The reaction mixture was concentrated to provide 2-(N-(3,4-dimethoxybenzyl)-4-methylphenyl-sulfonamido)acetyl chloride as a yellow solid (50 g).

Step 5. Into a 500 mL 3-necked round bottom flask purged and maintained with an inert atmosphere of nitrogen and maintained at −78° C. in a bath of liquid N₂ was added 2-(N-(3,4-dimethoxybenzyl)-4-methylphenylsulfonamido)acetyl chloride (50 g, 100.55 mmol), DCM (300 mL) and AlCl₃ (53 g, 398.50 mmol). The resulting solution was stirred for 4 hr at −78° C. and then for 4 hr at −10° C., followed by the dropwise addition of 10% aqueous HCl/ice at −10° C. with stirring over 30 min. The resulting solution was extracted with CH₂Cl₂, and the combined organic fractions were washed with H₂O and brine, dried over anhydrous Na₂SO₄, and concentrated to provide 35 g of crude 6,7-dimethoxy-2-tosyl-2,3-dihydroisoquinolin-4(1H)-one as red oil.

Step 6. A mixture of 6,7-dimethoxy-2-tosyl-2,3-dihydroisoquinolin-4(1H)-one (20 g, 55.40 mmol) in saturated aqueous NaHCO₃ (150 mL) and EtOH (30 mL) was refluxed overnight. The mixture was concentrated and extracted with EtOAc. The organic fraction was washed with H₂O and brine, dried over anhydrous Na₂SO₄, and concentrated. The residue was purified by silica gel chromatography using 5:1 (v/v) CH₂Cl₂/MeOH as an eluant to provide 1 g of 6,7-dimethoxyisoquinolin-4-ol as a brown solid.

Step 7. Into a 250 mL 3-necked round bottom flask purged and maintained with an inert atmosphere of nitrogen and maintained at 0° C. was added 6,7-dimethoxyisoquinolin-4-ol (1.35 g, 5.27 mmol), DCM (200 mL), Et₃N (3.4 g, 33.66 mmol) and Tf₂O (2.4 g, 8.51 mmol). The resulting solution was stirred for 30 min at 0° C. and reaction was monitored by TLC (EtOAc/PE (1:1, v/v)). The resulting mixture was washed with H₂O and brine, dried over anhydrous Na₂SO₄, and concentrated. The residue was purified by silica gel chromatography using 1:6 (v/v) EtOAc/PE as an eluant to provide 6,7-dimethoxyisoquinolin-4-yl trifluoromethanesulfonate as a yellow solid (650 mg). ¹H NMR (400 Hz, DMSO) δ 3.97(3H,s), 3.99(3H,s), 7.20(1H,s), 7.73(1H,s), 8.52(1H,s), 9.19(1H,s). LCMS [M+H]⁺ calcd for C₁₂H₁₁F₃NO₅S 338, found 338.

### Example 4

### Synthesis of 6,7-dimethoxy-1-(6-morpholin-4-ylpyridin-3-yl)isoquinoline

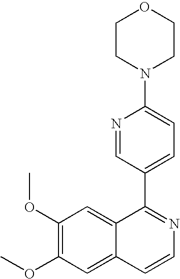

A mixture of 1-bromo-6,7-dimethoxyisoquinoline (50.2 mg, 0.187 mmol), 4-[5-(4,4,5,5-tetramethyl-[1,3,2]dioxaborolan-2-yl)-pyridin-2-yl]morpholine (140 mg, 0.482 mmol), bis(triphenylphosphine)palladium(II) chloride (27.1 mg, 0.039 mmol), and 2.0 M sodium carbonate in water (40 μL) in 1,2-dimethoxyethane:water:ethanol (7:3:2, v/v/v) (901 μL) was irradiated in a microwave reactor at 140° C. for 5.0 min. The reaction mixture was filtered through a plug of celite, and rinsed with methanol. The product was purified by rotary chromatography using a gradient from 0 to 10 v% MeOH in chloroform as an eluant to provide 45 mg of 6,7-dimethoxy-1-(6-morpholin-4-ylpyridin-3-yl)isoquinoline. ¹H NMR (300 MHz CDCl₃) δ 8.52 (d, 1H), 8.39 (d, 1H), 7.88 (dd, 1H), 7.40 (d, 1H), 7.36 (s, 1H), 7.05 (s, 1H), 6.75 (d, 1H), 3.98 (s, 3H), 3.86 (s, 3H), 3.82-3.79 (m, 4H), and 3.58-3.54 (m, 4H). LC/MS Method (2080_{—}8min), retention time, 2.01 min, M+H=352.1.

### Example 5

### Synthesis of 6,7-dimethoxy-4-[2-(4-methoxyphenyl)morpholin-4-yl]quinoline

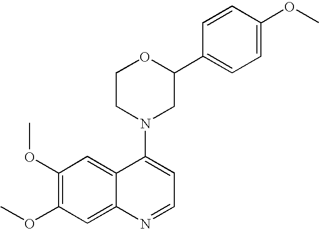

A mixture of 4-bromo-6,7-dimethoxyquinoline (60 mg, 0.22 mmol), 2-(4-methoxyphenyl)morpholine (51 mg, 0.26 mmol), 2-dicyclohexylphosphino-2′,3′,6′-tri-isopropyl-1,1′-biphenyl (13 mg, 0.026 mmol), and sodium tert-butoxide (64.5 mg, 0.67 mmol), tris(dibenzylideneacetone)dipalladium (0) (13 mg, 0.014 mmol) in and tetrahydrofuran (3.8 mL) was irradiated in a microwave reactor to 135° C. for 20 min. The reaction mixture was then filtered through a plug of celite. After concentration, the crude product was purified by column chromatography using a gradient from 0 to 5 v% MeOH in 1:1 (v/v) EtOAc/hexane and DMEA 0.3 v%, followed by preparative HPLC over a C 18 reverse phase column using a gradient from 5 v% to 60 v% acetonitrile in water with 0.1 v% formic acid over 8 min with a flow rate of 40 mL/min to provide 6,7-dimethoxy-4-[2-(4-methoxyphenyl)morpholin-4-yl]quinoline as a yellow gum (28 mg, 34%). ¹H NMR (300 MHz CDCl₃) δ 8.60 (d, 1H), 7.80 (s, 1H), 7.36-7.25 (m, 3H), 6.94-6.89 (m, 3H), 4.82 (dd, 1H), 4.30-4.26 (m, 1H), 4.18-4.13 (m, 1H), 4.09 (s, 3H), 4.05 (s, 3H), 3.82 (s, 3H), 3.74-3.64 (m, 2H), 3.33 (dt, 1H), 3.12 (dd, 1H). LC/MS Method (2080_{—}8min), retention time, 3.94 min, M+H=381.2.

### Example 6

### Synthesis of 1′-(6,7-dimethoxyisoquinolin-1-yl)-1,3′-bipiperidin-2-one

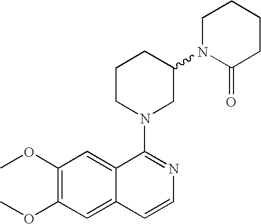

To a flame-dried microwave tube under argon was added 1-bromo-6,7-dimethoxyisoquinoline (49.9 mg, 0.186 mol), 3-(N-delta-valerolactam)piperidine hydrochloride (50.0 mg, 0.228 mmol), tris(dibenzylideneacetone)dipalladium(0) (8.6 mg, 0.0094 mmol), 9,9-dimethyl-4,5-bis(diphenylphosphino)xanthene (11.5 mg, 19.9 mmol), sodium tert-butoxide (44.4 mg, 0.462 mol), and toluene (0.5 mL). The dark brown suspension was stirred at 50° C. overnight. The reaction mixture was filtered through celite, rinsed with ˜30 mL of 10 v% MeOH in DCM, and concentrated (rotovap). The compound was purified on a C18 preparative HPLC column (30×100 mm) using acetonitrile:water (with 0.1 v% formic acid) in a gradient from 20 v% CH₃CN to 80 v% CH₃CN at a flow rate of 45 mL/min. The fractions were monitored at a wavelength of 352 nm and the product had a retention time of 2 to 3 min. The material was loaded onto an SCX column, rinsed with one column volume of MeOH, and eluted with 2.0 M ammonia in methanol (8 mL). Concentration of the solvent provided 1′-(6,7-dimethoxyisoquinolin-1-yl)-1,3′-bipiperidin-2-one as a white solid (7.3 mg). ¹H NMR (300 MHz CDCl₃) δ 8.05 (d, 1H), 7.49 (s, 1H), 7.17 (d, 1H), 7.02 (s, 1H), 5.07 (tt, 1H), 4.12 (s, 3H), 4.01 (s, 3H), 3.64 (d, 2H) 3.33-3.23 (m, 2H), 2.91-2.75 (m, 2H), 2.50-2.30 (m, 2H), 2.05-1.88 (m, 3H), 1.83-1.67 (m, 5H). LCMS: Retention time=2.83, M+H=370.2.

### Example 7

### Synthesis of 1′-(6,7-dimethoxyguinolin-4-yl)-1,3′-bipiperidin-2-one

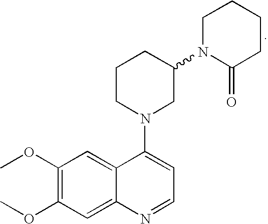

Into a flame-dried 5 mL microwave tube under argon was added 4-bromo-6,7-dimethoxyquinoline (73.3 mg, 0.273 mmol), 3-(N-delta-valerolactam)piperidine hydrochloride (81.8 mg, 0.374 mmol), tris(dibenzylideneacetone)dipalladium(0) (12.3 mg, 0.0134 mmol), 9,9-dimethyl-4,5-bis(diphenylphosphino)xanthene (16.6 mg, 0.0287 mmol), sodium tert-butoxide (74.2 mg, 0.772 mmol), and toluene (0.7 mL). The yellow suspension was stirred at 60° C. for 65 hr, filtered through celite, rinsed with ˜30 mL of 10 v% MeOH in DCM, and concentrated (rotovap). The crude product was purified on a C 18 preparative HPLC column (30×100 mm) using 15 v% CH₃CN in water (with 0.1 v% formic acid) for 5 min, and then using a gradient from 15 v% CH₃CN to 80 v% CH₃CN over 2 min at a flow rate of 45 mL/min. Fractions were monitored at a wavelength of 357 nm and the product was collected from 3.25 to 5.25 min. The material was loaded onto an SCX column (0.71 g), rinsed with one column volume of MeOH, and eluted with 2.0 M ammonia in methanol (8 mL). Removal of the solvent (rotovap) and dried under reduced pressure provided 1′-(6,7-dimethoxyquinolin-4-yl)-1,3′-bipiperidin-2-one as a white solid (51.1 mg).

### Example 8

### Synthesis of 1-(6-fluoropyridin-3-yl)-6,7-dimethoxyisoquinoline

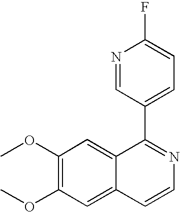

To a mixture of 1-bromo-6,7-dimethoxyisoquinoline (0.4834 g, 1.803 mmol) and tetrakis(triphenylphosphine)palladium (0.1152 g, 0.09015 mmol) in 1,2-dimethoxyethane (30 mL) was added 6-fluoropyridin-3-ylboronic acid (0.2849 g, 1.983 mmol) with stirring. A solution of cesium carbonate (1.6792 g, 4.868 mmol) in water (10 mL) was then added. The resulting mixture was stirred at 80° C. for 3 hr. The reaction was monitored by LCMS. Upon completion, the reaction was allowed to cool to room temperature. The solution was moved to a seperatory funnel, and water and ethyl acetate was added. The aqueous layer was extracted ethyl acetate three times. The combined organic layers were washed with water and saturated sodium chloride solution, dried with anhydrous magnesium sulfate, filtered, and concentrated. The crude product was adsorbed onto a plug of silica gel and chromatographed through a Biotage pre-packed silica gel column (40S), eluting with a gradient of 10 v% to 60 v% ethyl acetate in hexane, to provide 1-(6-fluoropyridin-3-yl)-6,7-dimethoxyisoquinoline (0.5 g).

### Example 9

### Synthesis of 5-(6,7-dimethoxyisoquinolin-1-yl)-N-isopropylpyridin-2-amine

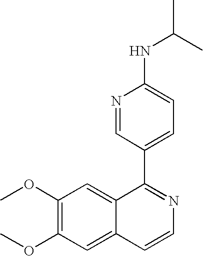

A mixture of 1-(6-fluoropyridin-3-yl)-6,7-dimethoxyisoquinoline (0.0580 g, 0.204 mmol) and propan-2-amine in 2mL DMSO (0.174 ml, 2.04 mmol) was stirred at 90° C. overnight. The reaction was monitored by LCMS. Upon completion, the reaction mixture was allowed to cool to room temperature. The solution was moved to a seperatory funnel and DI water and EtOAc was added. The aqueous layer was extracted EtOAc three times. The combined organic layers were washed with water and brine, dried with anhydrous MgSO₄, filtered, and concentrated. The crude product was adsorbed onto a plug of silica gel and chromatographed through a Biotage pre-packed silica gel column (40S), eluting with a gradient of 1 v% to 5 v% MeOH in CH₂Cl₂, to provide 5-(6,7-dimethoxyisoquinolin-1-yl)-N-isopropylpyridin-2-amine (0.0356 g, 0.110 mmol).

### Example 10

### Synthesis of 5-(6,7-dimethoxvisoguinolin-1-yl)-N-ethyl-N-propylpyridin-2-amine

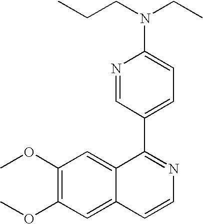

To a microwave reaction vessel was added 1-(6-fluoropyridin-3-yl)-6,7-dimethoxyisoquinoline (0.0792 g, 0.28 mmol) in 2 mL DMSO. N-Ethylpropan-1-amine (0.34 ml, 2.8 mmol) was added and allowed to stir at 90° C. overnight. Reaction was monitored by LCMS. An additional 10 equivalents of N-ethylpropan-1-amine was added and allowed to stir overnight. When the reaction was recorded to be 70% complete by LCMS, the reaction was allowed to cool to room temperature. The solution was moved to a separatory funnel and DI water and EtOAc was added. The aqueous layer was extracted with EtOAc. The combined organic layers were washed with water, brine, dried with MgSO₄, filtered, and concentrated. The crude product was adsorbed onto a plug of silica gel and chromatographed through a Biotage pre-packed silica gel column (40S), eluting with a gradient of 1% to 5% MeOH in CH₂Cl₂, to provide 5-(6,7-dimethoxyisoquinolin-1-yl)-N-ethyl-N-propylpyridin-2-amine (0.0700 g, 0.20 mmol).

### Example 11

### Synthesis of 1-(6-((2S,6R)-2,6-dimethylmorpholino)pyridin-3-yl)-6,7-dimethoxyisoguinoline

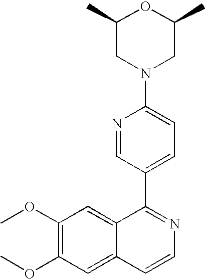

To a microwave reaction vessel was added 1-(6-fluoropyridin-3-yl)-6,7-dimethoxyisoquinoline (0.0733 g, 0.26 mmol) in 2 mL DMSO. Cis-2,6-dimethylmorpholine (0.320 ml, 2.6 mmol) was added and allowed to stir at 90° C. overnight. Reaction was monitored by LCMS. Upon completion, the reaction was allowed to cool to room temperature. The solution was moved to a seperatory funnel and DI water and EtOAc was added. The aqueous layer was extracted EtOAc three times. The combined organic layers were washed with water, brine, dried with MgSO₄, filtered, and concentrated. The crude product was adsorbed onto a plug of silica gel and chromatographed through a Biotage pre-packed silica gel column (40S), eluting with a gradient of 1 v% to 5 v% MeOH in CH₂Cl₂, to provide 1-(6-((2S,6R)-2,6-dimethylmorpholino)pyridin-3-yl)-6,7-dimethoxyisoquinoline (0.0765 g, 0.20 mmol).

### Example 12

### Synthesis of 6,7-dimethoxy-4-(2-methylbenzo[d]thiazol-5-yl)isoguinoline

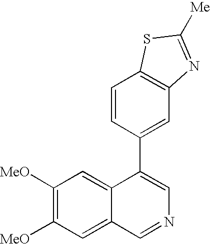

To a solution of 6,7-dimethoxyisoquinolin-4-yl trifluoromethanesulfonate (165 mg, 489 μmol) in dimethoxyethane was added 2-methyl-5-(4,4,5,5-tetramethyl-1,3,2-dioxaborolan-2-yl)benzo[d]thiazole (188 mg, 685 ∞mol), followed by trans-dichlorobis(triphenyl-phosphine)palladium (ii) (17 mg, 24 μmol). An aqueous solution of cesium carbonate (430 mg, 1321 μmol) in H₂O (5.2 mL) was then added and the mixture was heated to 80° C. for two hr. LCMS analysis showed complete consumption of the starting material. The mixture was cooled to room temperature, diluted with ethyl acetate and H₂O, the layers were separated and the aqueous was extracted with ethyl acetate three times. The combined organics were washed with brine, dried over anhydrous Na₂SO₄, filtered, and concentrated. The residue was purified by Biotage, 25 m column, 20-100% EA/DCM to yield the title compound.

### Biological Examples

### Example 13

### mPDE 10A7 Enzyme Activity and Inhibition

Enzyme Activity. To analyze the enzyme activity, 5 μL of serial diluted mPDE10A7 containing lysate were incubated with equal volumes of diluted (100-fold) fluorescein labeled cAMP or cGMP for 30 min in MDC HE 96-well assay plates (Molecular Devices Corp., Sunnyvale Calif.) at room temperature. Both the enzyme and the substrates were diluted in the following assay buffer: Tris/HCl (pH 8.0) 50 mM, MgCl₂ 5 mM, 2-mercaptoethanol 4 mM, and BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP assay kit (Molecular Devices) was used to assess enzyme properties of mPDE10A7. Data were analyzed with SOFTMAX PRO software (Molecular Devices).

Enzyme Inhibition. To check the inhibition profile, 10 μL of serial diluted compounds were incubated with 30 μl of diluted PDE enzymes in a 96-well polystyrene assay plate for 30 min at room temperature. After incubation, 5 μL of the compound-enzyme mixture were aliquoted into a MDC HE black plate, mixed with 5 μL of 100-fold diluted fluorescein labeled substrates (cAMP or cGMP), and incubated for 30 min at room temperature. The reaction was stopped by adding 20 μL of diluted binding reagents and counted in an Analyst GT for fluorescence polarization. The data were analyzed with SoftMax Pro.

### Example 14

### Apomorphine Induced Deficits in Prepulse Inhibition of the Startle Response in Rats an in vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g., the reflex activity of the rats is measured. The reflex of the rats to the burst of noise may be attenuated by preceding the startle stimulus with a stimulus of lower intensity, at 3 to 12 db above background (65 db), which attenuates the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine reduces the inhibition of the startle reflex produced by the prepulse. Antipsychotic drugs such as haloperidol prevents apomorphine from reducing the prepulse inhibition of the startle reflex. This assay can be used to test the antipsychotic efficacy of PDE10 inhibitors, as they reduce the apomorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 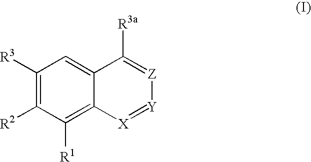
or an individual stereoisomer, a mixture of stereoisomers, or a pharmaceutically acceptable salt or solvate thereof, wherein:
X is nitrogen and Y and Z are each —CH═ or one of Y and Z is nitrogen and the other is —CH═ and X is —CR═ (where R is hydrogen, alkyl, halo, or cyano);
R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, cycloalkyl, cycloalkyloxy, cycloalkylalkyloxy, hydroxyalkyl, hydroxyalkyloxy, alkoxyalkyl, alkoxyalkyloxy, -(alkylene)NR¹³R¹⁴ and —O-(alkylene)NR¹⁵R¹⁶ (where R¹³, R¹⁴, R¹⁵, and R¹⁶ are independently hydrogen or alkyl), wherein one or two carbon atoms in the alkyl chain in hydroxyalkyl, hydroxyalkyloxy, alkoxyalkyl, alkoxyalkyloxy, -(alkylene)NR¹³R¹⁴ or —O-(alkylene)NR¹⁵R¹⁶ are optionally replaced by one to two oxygen or nitrogen atom(s), and provided that at least one of R′, R², and R³ is not hydrogen; and
R^{3a} is aryl, heteroaryl, or heterocyclyl ring substituted with:
R⁴, where R⁴ is hydrogen, alkyl, halo, haloalkyl, haloalkoxy, cycloalkyl, cycloalkenyl, cycloalkylalkyl, aryl, aralkyl, heteroaryl, heteroaralkyl, heterocyclyl, heterocyclylalkyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹, and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶, where R⁵ and R⁶ are independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl, and provided that at least one of R⁴, R⁵, and R⁶ is not hydrogen;
wherein the aromatic or alicyclic ring in R⁴, R⁵, R⁶, and R⁷ is optionally substituted with one to three substitutents independently selected from R^{a}, R^{b}, and R^{c}, which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl or optionally substituted heterocyclyl; and additionally substituted with one or two substitutents independently selected from R^{d} and R^{e} where R^{d} and R^{e} are hydrogen or fluoro;
provided that:
(a) when R is hydrogen, R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, and cycloalkyl, and:
(i) R^{3a} is pyrrolidin-1-yl, then R⁴ is not —X¹R⁷ where X¹ is —O— and R⁷ is substituted or unsubstituted aryl or heteroaryl;
(ii) R^{3a} is piperidin-1-yl, where one of R⁴, R⁵ and R⁶ is hydrogen and another of R⁴, R⁵ and R⁶ is substituted or unsubstituted aryl or heteroaryl, then the remaining member of R⁴, R⁵ and R⁶ is not hydrogen; alkyl; carboxy; cyano; hydroxyl; alkoxy; —COR′, —CONR′R″ or —NR′R″ (where R′ and R″ are independently hydrogen, alkyl, or unsubstituted aryl); or —NHCOR¹ (where R¹ is alkyl or unsubstituted aryl); or
(iii) R^{3a} is piperidin-1-yl, where two of R⁴, R⁵ and R⁶ are hydrogen, then remaining of R⁴, R⁵ and R⁶ is not —COR¹ (where R¹ is alkyl or unsubstituted aryl), —COOR¹ (where R¹ is alkyl or unsubstituted aryl), —CONR′R″, —NR′R″ or —NHCOR¹ (where each R¹¹is hydrogen, alkyl, or unsubstituted aryl, and each R¹ is unsubstituted aryl);
(b) when R is hydrogen, R¹, R², and R³ are each independently selected from hydrogen, alkyl, alkoxy, halo, haloalkyl, haloalkoxy, cyano, hydroxy, carboxy, alkoxycarbonyl, amino, alkylamino, dialkylamino, alkylcarbonyl, and cycloalkyl, then:
(i) R^{3a} is not substituted or unsubstituted 1,2,3,4-tetrahydroisoquinolin-3-yl or 1,2,3,4-tetrahydroisoquinolin-2-yl; or
(ii) R^{3a} is not monosubstituted or disubstituted pyrrolidinyl where the one or two substituents are alkyl;
(c) when R is hydrogen, alkyl, or alkoxy, R¹, R², and R³ are independently hydrogen, halo, haloalkyl, alkyl, alkoxy, carboxy, hydroxymethyl or hydroxy, and R^{3a} is aryl, then one of R⁴, R⁵, and R⁶ is an aromatic or alicyclic ring or a group that contains an aromatic or alicyclic ring provided that the aromatic or alicyclic ring is not phenyl (optionally substituted with one, two or three substitutents independently selected from cyano, halo, —CONH₂ and haloalkyl), benzyl, benzyloxy, 1H-benzimidazol-2-ylthio, 1H-benzimidazol-2-ylsulfinyl, pyridinyl (optionally substituted with halo or —CONH₂), pyrimidinyl, or morpholin-4-yl-carbonyl;
(d) when R is hydrogen, R¹, R², and R³ are independently hydrogen, halo, alkoxy, or hydroxy, and R^{3a} is heteroaryl, then the heteroaryl ring is not phthalazin-1-yl optionally substituted with R⁴, R⁵, and R⁶where R⁴ is alkyl and R⁵ and R⁶ are alkoxy;
isoquinolinyl optionally substituted with one or two substituents selected from alkoxy and hydroxy;
1H-indolyl optionally substituted with R⁴, R⁵, and R⁶ where R⁴ is hydrogen, one of R⁵ and R⁶ is hydrogen, alkyl, or alkoxy, and the other of R⁵ and R⁶ is alkyl, alkoxy, haloalkyl, dialkylaminoalkyl, or hydroxyalkyl;
benzo[c]isoxazolyl optionally substituted with R⁴, R⁵, and R⁶ where one of R⁴, R⁵, and R⁶ is hydrogen and the other two of R⁴, R⁵, and R⁶ are independently selected from alkoxy, aryl, or benzyloxy;
1H-indazolyl optionally substituted with one or two alkoxy or hydroxy;
pyrrolyl substituted with R⁴, R⁵, and R⁶ where one of R⁴, R⁵, and R⁶ is hydrogen or alkyl and the other two of R⁴, R⁵, and R⁶ are phenyl optionally substituted with one or two alkoxy;
thienyl optionally substituted with halo; or
pyrazolyl optionally substituted with R⁴, R⁵, and R⁶ where R⁴ is hydrogen, one of R⁵ and R⁶ is alkoxycarbonyl and the other of R⁵ and R⁶ is alkoxyalkyl;
(e) when R is hydrogen or alkoxy, R¹, R², and R³ are independently hydrogen, halo, alkyl, haloalkyl, haloalkoxy, alkoxy, carboxy, hydroxymethyl or hydroxy, then R^{3a} is not
monosubstituted piperazinyl [wherein the substitutent on piperazinyl ring is alkyl, alkoxycarbonyl, phenyl, —COR′ (where R′ is alkyl; or piperidinyl or pyrrolidinyl each optionally substituted with one or two substituents each independently selected from alkyl or hydroxyl), hydroxyalkyl, —CONHR′ (where R′ is phenyl substituted with fluoro or phenoxy), 1H-benzo[d]imidazol-2(3H)-one optionally substituted with alkyl, or 3,4-dihydroquinolinyl-2(1H)-one];
substituted or unsubstituted benzimidazolyl, 1,2,3,4-tetrahydroisoquinolinyl, isoquinolinyl, isobenzofuranyl-1(3H)-one, 1,2,3-oxadiazolyl-5(2H)-one, 1,3,4-oxadiazolyl-2(3H)-one, 2,3-dihydrobenzo[b][1,4]dioxinyl, benzo[d][1,3]dioxolyl, 1,2,4,5,6,7-hexahydropyrazolo[1,5-a]pyridinyl, 1,2-dihydropyrazolo[1,5-a]pyridinyl, H-pyrazolo[1,5-a]pyridinyl, 5,6-dihydro-4H-pyrrolo[1,2-b]pyrazolyl, benzisoxazolyl, 1,1-dioxo-3H-benzo[c][1,2]oxathiolyl, benzofuranyl-2(3H)-one, (Z)-1H-benzo[e][1,4]diazepinyl-2(3H)-one, 1,3a-dihydropyrazolo[1,5-a]pyridinyl, oxazolyl-2(3H)-one, naphthyl, or imidazo[5,1-a]isoquinolinyl;
mono or disubstituted piperidinyl (where one substituent is hydrogen or hydroxyl, and the other substitutent is alkoxy, hydroxyl, carboxy, or 1H-benzo[d]imidazol-2(3H)-one optionally substituted with alkyl); or pyrrolidinyl optionally substituted with alkyl or alkoxy;
(f) when X is N, then at least two of R¹, R² and R³ are not simultaneously hydrogen; and
(g) the compound is not a salt of (a)-(f).

2. The compound of claim 1, wherein X is nitrogen and Y and Z are —CH═.

3. The compound of claim 1, wherein Y is nitrogen and X and Z are —CH═.

4. The compound of claim 1, wherein Z is nitrogen and X and Y are —CH═.

5. The compound of claim 2, wherein R¹ is hydrogen and R² and R³ are independently alkoxy.

6. The compound of claim 2, wherein R¹ is hydrogen, one of R² and R³ is alkoxy, and the other is alkyl.

7. The compound of claim 2, wherein R¹ is hydrogen, one of R² and R³ is alkoxy and the other is halo or haloalkoxy.

8. The compound of claim 3, wherein R¹ is hydrogen and R² and R³ are independently alkoxy.

9. The compound of claim 3, wherein R¹ is hydrogen, one of R² and R³ is alkoxy, and the other is alkyl.

10. The compound of claim 3, wherein R¹ is hydrogen, one of R² and R³ is alkoxy, and the other is halo or haloalkoxy.

11. The compound of claim 4, wherein R¹ is hydrogen, and R² and R³ are independently alkoxy.

12. The compound of claim 4, wherein R¹ is hydrogen, one of R² and R³is alkoxy, and the other is alkyl.

13. The compound of claim 4, wherein R¹ is hydrogen, one of R² and R³ is alkoxy and the other is halo or haloalkoxy.

14. The compound of claim 2, wherein R¹ is hydrogen, R² and R³ are independently alkoxy, and R^{3a} is a ring of formula: 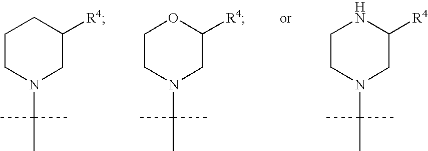
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

15. The compound of claim 2, wherein R¹ is hydrogen, one of R² and R³is alkoxy and the other is alkyl, and R^{3a} is a ring of formula: 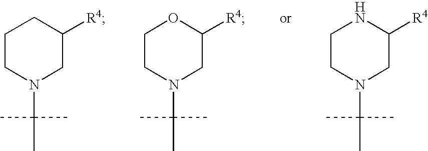
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

16. The compound of claim 2 wherein R¹ is hydrogen, one of R² and R³ is alkoxy and the other is halo or haloalkoxy, and R^{3a} is a ring of formula: 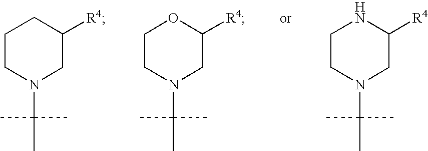
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

17. The compound of claim 3, wherein R¹ is hydrogen, R² and R³ are each independently alkoxy, and R^{3a} is a ring of formula: 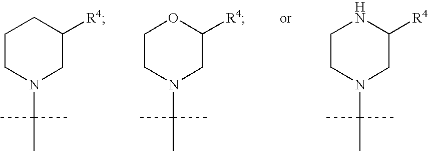
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

18. The compound of claim 3, wherein R¹ is hydrogen, one of R² and R³ is alkoxy and the other is alkyl, and R^{3a} is a ring of formula: 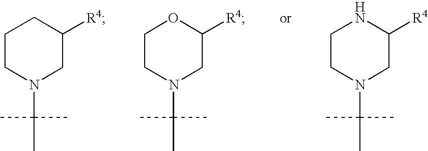
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

19. The compound of claim 3, wherein R¹ is hydrogen, one of R² and R³ is alkoxy and the other is halo or haloalkoxy, and R^{3a} is a ring of formula: 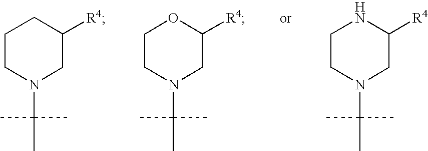
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

20. The compound of claim 4, wherein R¹ is hydrogen, R² and R³ are each independently alkoxy, and R^{3a} is a ring of formula: 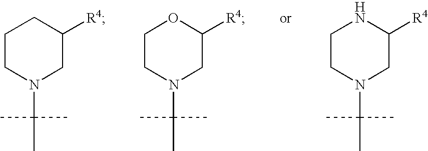
where R⁴ is phenyl, heteroaryl, or six membered saturated heterocyclyl each optionally substituted with R^{a}, R^{b} and R^{c} and where the rings are substituted, including the hydrogen atom on the —NH— group within the ring, with R⁵ and R⁶.

21. The compound of claim 4, wherein R¹ is hydrogen, one of R² and R³ is alkoxy and the other is alkyl, and R^{3a} is a ring of formula: 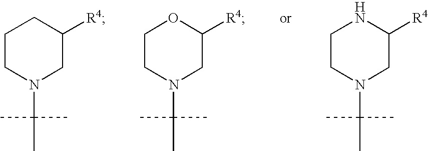
where R⁴ is phenyl, heteroaryl, or six-membered saturated heterocyclyl each optionally substituted with R^{a}, R^{b} and R^{c} and where the rings are substituted, including the hydrogen atom on the —NH— group within the ring, with R⁵ and R⁶.

22. The compound of claim 4, wherein R¹ is hydrogen, one of R² and R³ is alkoxy and the other is halo or haloalkoxy, and R^{3a} is a ring of formula: 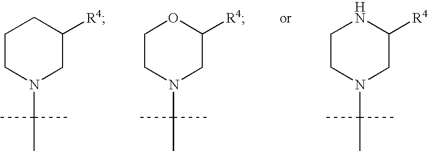
where R⁴ is phenyl, heteroaryl, or six membered saturated heterocyclyl each optionally substituted with R^{a}, R^{b} and R^{c} and where the rings are substituted, including the hydrogen atom on the —NH— group within the ring, with R⁵ and R⁶.

23. The compound of claim 2, wherein R¹ is hydrogen, R² and R³ are each independently alkoxy, and R^{3a} is a ring of formula:

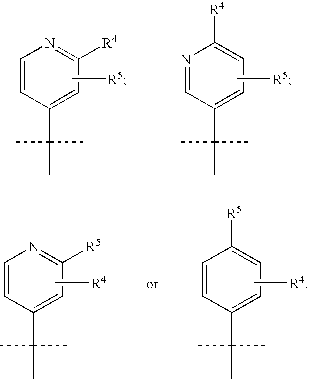

24. The compound of claim 2, wherein R¹ is hydrogen, one of R² and R³ is alkoxy and the other is alkyl, and R^{3a} is a ring of formula:

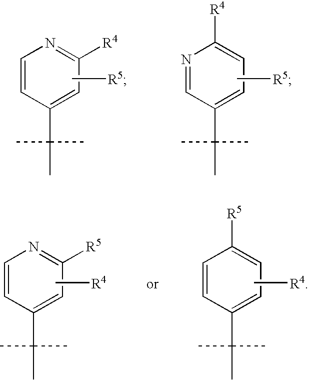

25. The compound of claim 2, wherein R¹ is hydrogen, one of R² and R³ is alkoxy and the other is halo or haloalkoxy, and R^{3a} is a ring of formula:

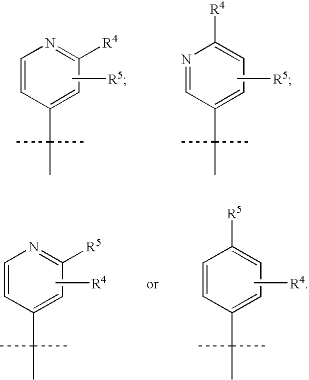

26. The compound of claim 3, wherein R¹ is hydrogen, R² and R³ are independently alkoxy, and R^{3a} is a ring of formula:

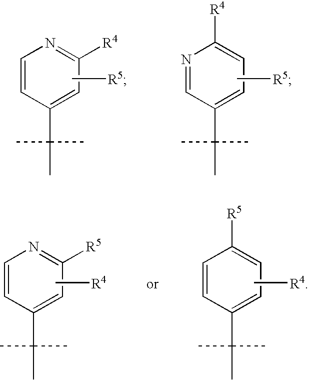

27. The compound of claim 3, wherein R¹ is hydrogen, one of R² and R³ is alkoxy and the other is alkyl, and R^{3a} is a ring of formula:

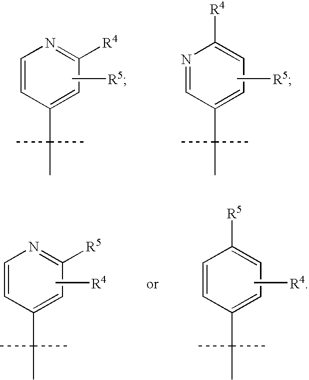

28. The compound of claim 3, wherein R¹ is hydrogen, one of R² and R³ is alkoxy and the other is halo or haloalkoxy, and R^{3a} is a ring of formula:

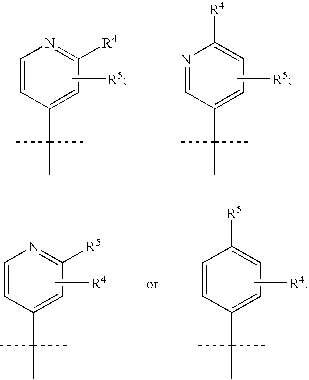

29. The compound of claim 4, wherein R¹ is hydrogen, R² and R³ are each independently alkoxy, and R^{3a} is a ring of formula:

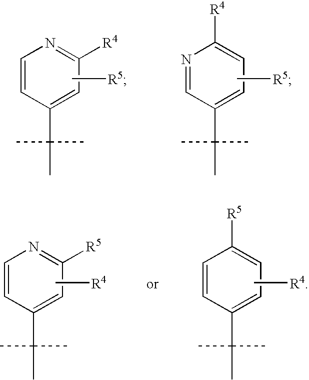

30. The compound of claim 4, wherein R¹ is hydrogen, one of R² and R³ is alkoxy and the other is alkyl, and R^{3a} is a ring of formula:

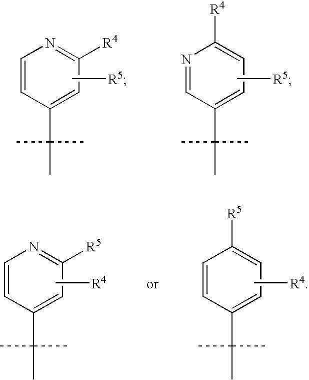

31. The compound of claim 4, wherein R¹ is hydrogen, one of R² and R³ is alkoxy and the other is halo or haloalkoxy, and R^{3a} is a ring of formula:

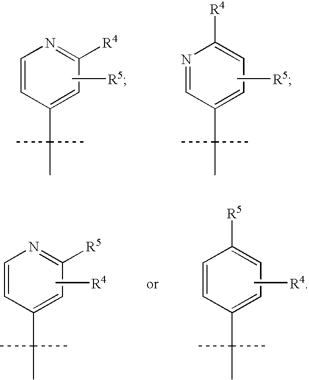

32. The compound of claim 1, wherein R^{3a} is a ring of formula:

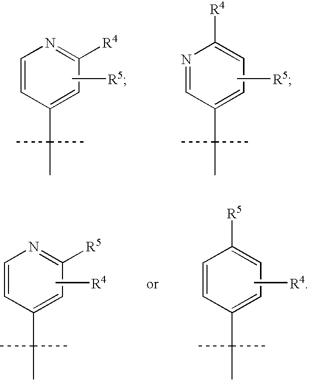

33. The compound of claim 1, wherein R¹ is hydrogen, R² and R³ are each independently alkoxy, alkyl, haloalkoxy, or halo, and R^{3a} is a ring of formula: 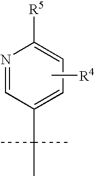
where R⁵ is heterocyclyl, monosubstituted or disubstituted amino wherein the aromatic or alicyclic rings in R⁵ are optionally substituted, and R⁴ is hydrogen, alkyl, or halo.

34. The compound of claim 2, wherein R¹ is hydrogen, R² and R³ are each independently alkoxy, alkyl, haloalkoxy, or halo, and R^{3a} is 5-, 6-, 7-, or -8-azaindolyl or benzthiazolyl, substituted with R⁴, R⁵, or R⁶.

35. A pharmaceutical composition comprising a compound of claim 1 and a pharmaceutically acceptable expicient.

36. A method of treating a disorder or disease treatable by inhibition of PDE10 enzyme in a patient which method comprises administering to the patient a pharmaceutical composition comprising a compound of claim 1 and a pharmaceutically acceptable expicient.

37. The method of claim 36, wherein the disease is schizophrenia, bipolar disorder, or obsessive-compulsive disorder.
